SEQUENCE LISTING (2 sequences)

SEQ ID NO: 1 - protein, 167 aa, Artificial Sequence
  Synthesized Sequence
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 2 - protein, 227 aa, Artificial Sequence
  Synthesized Sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0273768 A1
(43) Pub. Date: Sep. 1, 2022

(54) INTRANASAL LEPTIN COMPOSITIONS AND METHODS OF USE THEREOF FOR PREVENTION OF OPIOID INDUCED RESPIRATORY DEPRESSION IN OBESITY

(71) Applicants: The George Washington University, Washington, DC (US); Johns Hopkins University, School of Medicine, Baltimore, MD (US)

(72) Inventors: David MENDELOWITZ, Vienna, VA (US); Vsevolod POLOTSKY, Pikesville, MD (US); Carla FREIRE, Baltimore, MD (US)

(21) Appl. No.: 17/747,905

(22) Filed: May 18, 2022

Related U.S. Application Data
(63) Continuation of application No. PCT/US2020/060560, filed on Nov. 13, 2020.
(60) Provisional application No. 62/937,708, filed on Nov. 19, 2019.

(51) Int. Cl. A61K 38/22; A61P 25/04

(52) U.S. Cl. CPC A61K 38/2264; A61P 25/04; A61P 11/00

(57) ABSTRACT

The present disclosure generally relates to compositions and methods of treating opioid-induced respiratory depression in a subject in need thereof, the method comprising administering leptin.

### CROSS REFERENCE TO RELATED APPLICATION

This application is a continuation of PCT Application No. PCT/US2020/060560, filed Nov. 13, 2020, which claims the benefit of U.S. Provisional Application No. 62/937,708 filed on Nov. 19, 2019, the disclosures of which are hereby incorporated by reference in its entirety.

### GOVERNMENT RIGHTS

This invention was made with government support under HL128970 awarded by the National Institutes of Health. The government has certain rights in the invention.

### FIELD

The present disclosure generally relates to compositions and methods of treating opioid-induced respiratory depression in a subject in need thereof, the method comprising administering leptin.

### INCORPORATION BY REFERENCE OF SEQUENCE LISTING PROVIDED ELECTRONICALLY

An electronic version of the Sequence Listing is filed herewith, the contents of which are incorporated by reference in their entirety. The electronic file is 3.84 kilobytes in size, and titled GW046_091019-640336_SequenceListing_ST25.txt.

### BACKGROUND

Synthetic opioids, particularly fentanyl and fentanyl analogues such as carfentanil, are responsible for surging death rates from opiate overdose worldwide. The primary cause of death associated with opiates is opiate-mediated respiratory suppression (ORS). Obese subjects are particularly at increased risk of opioid-induced death as a majority of obese individuals have obstructive sleep apnea. Further, upper airway obstruction is common under sedation, especially in subjects with obstructive sleep apnea. Both obstructive sleep apnea and upper airway obstruction impedes respiratory function which can increase the risk of death associated from opiate-mediated respiratory suppression. With the rising rate of obesity, there is an increased demand for treatment of opioid overdose. Further increasing the urgency is the current response for opioid overdose, naloxone, is not a sufficient antidote for synthetic opioids which have extremely long half-lives.

### SUMMARY

Embodiments of the instant disclosure relate to novel compositions, methods and systems for opioid-induced respiratory depression and opioid overdose in a subject. In certain embodiments, compositions include nasal formulations including leptin or combination agents thereof are disclosed. In other embodiments, nasal formulations disclosed herein can include at least one adherence agent for prolonging nasal mucosal interaction of the nasal formulation containing leptin. In some embodiments, methods of treating opioid-induced respiratory depression and/or treating an opioid overdose in a subject using the compositions and nasal formulations disclosed herein are described.

An aspect of the present disclosure provides for compositions for treating opioid-induced respiratory depression in a subject in need thereof In various embodiments, compositions herein can include leptin. In some examples, a leptin included in compositions herein can be a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof. In various embodiments, the compositions here can include at least one pharmaceutical excipient. In various embodiments, the compositions here can further include naloxone.

In various embodiments, compositions herein can be formulated for intranasal administration. In some aspects, compositions herein can be an intranasal spray. In some other aspects, compositions herein can be an intranasal drop.

In various embodiments, compositions herein can include therapeutically effective amount of leptin. In some aspects, a therapeutically effective amount of leptin can increase the breathing rate of the subject in need thereof after administering the composition. In some other aspects, a therapeutically effective amount of leptin can increase upper airway patency of the subject in need thereof after administering the composition.

Another aspect of the present disclosure provides for methods of treating opioid-induced respiratory depression in a subject in need thereof In various embodiments, methods herein include administering leptin to a subject in need thereof. In some aspects, leptin can be administered intranasally to a subject in need thereof.

In various embodiments, methods of treating opioid-induced respiratory depression in a subject in need thereof can include a human subject. In some aspects, a subject in need thereof can be a human having a Body Mass Index (BMI) no less than about 30. In some other aspects, a subject in need thereof can be a human having a Body Mass Index (BMI) of about 25 to about 30. In still some other aspects, a subject in need thereof can be a human having a Body Mass Index (BMI) of no more than about 25. In some examples, a human subject can have leptin resistance. In some examples, a human subject can have obstructive sleep apnea.

In various embodiments, methods of treating opioid-induced respiratory depression in a subject in need thereof can increase breathing rate of the subject in need thereof after administering leptin compared to an untreated subject with identical disease condition and predicted outcome. In some examples, the breathing rate of the subject in need thereof can increase by at least 10% after administering leptin.

In various embodiments, methods of treating opioid-induced respiratory depression in a subject in need thereof can increase upper airway patency of the subject in need thereof after administering leptin compared to an untreated subject with identical disease condition and predicted outcome. In some examples, the upper airway patency of the subject in need thereof can increase by at least 10% after administering leptin.

In various embodiments, methods of treating opioid-induced respiratory depression in a subject in need thereof can improve obstructive sleep apnea of the subject in need thereof after administering leptin compared to an untreated subject with identical disease condition and predicted outcome. In some examples, the obstructive sleep apnea of the subject in need thereof can improve by at least 10% after administering leptin.

In various embodiments, methods of treating opioid-induced respiratory depression in a subject in need thereof can increase the tidal volume of the subject in need thereof after administering leptin compared to an untreated subject with identical disease condition and predicted outcome. In some examples, the tidal volume of the subject in need thereof can increase by at least 10% after administering leptin.

In various embodiments, methods of treating opioid-induced respiratory depression in a subject in need thereof can increase maximum inspiratory flow rate (Vᵢₘₐₓ) of the subject in need thereof after administering leptin compared to an untreated subject with identical disease condition and predicted outcome. In some examples, the maximum inspiratory flow rate (Vᵢₘₐₓ) of the subject in need thereof can increase by at least 10% after administering leptin.

In various embodiments, methods of treating opioid-induced respiratory depression in a subject in need thereof can increase minute ventilation of the subject in need thereof after administering leptin compared to an untreated subject with identical disease condition and predicted outcome. In some examples, the minute ventilation of the subject in need thereof can increase by at least 10% after administering leptin.

In various embodiments, leptin can be administered in combination with naloxone. In some aspects, administering a combination of leptin with naloxone can be synergistic. In some aspects, administering a combination of leptin with naloxone can be additive. In some aspects, administering leptin to a subject can replace the need for naloxone in the subject in need thereof. In some aspects, leptin can be administered in combination with at least one opioid. In some aspects, wherein leptin can be administered in combination with at least one opioid for up to 5 days following surgery. In some aspects, wherein leptin is administered immediately following an overdose of at least one opioid.

Another aspect of the present disclosure provides for methods of administering leptin intranasally to a subject in need thereof. In various embodiments, leptin can be administered intranasally at least once every 12 hours as needed to the subject in need thereof. In various embodiments, leptin can be administered intranasally at least once every 6 hours as needed to the subject in need thereof. In various embodiments, leptin can be administered intranasally at least once to the subject in need thereof. In various embodiments, leptin can be administered intranasally acutely to the subject in need thereof.

### BRIEF DESCRIPTION OF THE FIGURES

FIGS. 1A and 1B show graphs depicting respiratory response to administration of subcutaneous leptin in lean mice. FIG. 1A: graph showing leptin increased minute ventilation (V_{E}) in lean C57BL/6J mice treated with leptin. FIG. 1B: graph showing leptin increased hypoxic ventilatory response (HVR) in lean C57BL/6J mice treated with leptin.

FIGS. 2A and 2B show images depicting representative REM sleep recording in diet-induced obese (DIO) mice treated intranasally with either vehicle (FIG. 2A) or leptin (FIG. 2B).

FIGS. 2C-2E depict graphs measuring maximal inspiratory flow (Vᵢₘₐₓ) (FIG. 2C), minute ventilation (V_{E}) (FIG. 2D) and decreased oxygen desaturation index (ODI>4% from baseline) (FIG. 2E) in in diet-induced obese (DIO) mice treated intranasally (IN) with either vehicle or leptin and injected intraperitoneally (IP) with either vehicle or leptin.

FIGS. 2F-2L depict images of tissue samples from diet-induced obese (DIO) mice treated either intranasally (IN) with vehicle, intranasally (IN) with leptin, or injected intraperitoneally (IP) with leptin probed for phosphorylated STAT3 (pSTAT3) (shown in green). “CC” is the central canal; “NTS” is the nucleus of the solitary tract; “NA” is the nucleus ambiguous and “XII” is the hypoglossal nucleus region of the tissue sample. FIG. 2F: Dorsal medulla section from DIO mouse treated with IN leptin probed for pSTAT3. FIG. 2G: Dorsal medulla section from DIO mouse treated with IP leptin probed for pSTAT3. FIG. 2H: Rostral ventral lateral medulla (RVLM) section from DIO mouse treated with IN leptin probed for pSTAT3. FIG. 2I: RVLM section from DIO mouse treated with IP leptin probed for pSTAT3. FIG. 2J: medulla section from DIO mouse treated with IN vehicle probed for pSTAT3. FIG. 2K: medulla section from DIO mouse treated with IN leptin probed for pSTAT3. FIG. 2L: a graph depicting quantification of pSTAT3-positive cells in the hypothalamus and medulla blindly counted from multiple sections from four mice, averaged and presented as cells per section.

FIGS. 3A, 3K and 3B show images depicting representative REM sleep recordings in diet-induced obese (DIO) mice at baseline (FIG. 3A), treated intranasally with vehicle and morphine (FIG. 3K) or treated intranasally with leptin and morphine (FIG. 3L)

FIGS. 3B-3F depict graphs showing percent of obstructed breaths (IFL %; FIG. 3B), decreased maximal inspiratory flow (V_{Imax}; FIG. 3C), decreased minute ventilation during IFL (V_{E} IFL, FIG. 3D) non-flow limited breathing (V_{E} NFL; FIG. 3E), and increased apnea-hypopnea index (AHI; FIG. 3F) in diet-induced obese (DIO) mice at baseline (B), treated intranasally with vehicle and morphine (M+V), or treated intranasally with leptin and morphine (M+L).

FIGS. 3G-3J depict images of tissue samples of the central canal (CC), which encompasses the hypoglossal motoneurons innervating genioglossus from diet-induced obese (DIO) mice treated treated intranasally with leptin and morphine. The outlined area in FIG. 3J is shown magnified in FIGS. 3G-3I where FIG. 3G shows retrograde tracer Cholera Toxin B (CTB), FIG. 3H shows μ opiate receptors (MOR), and FIG. 3I shows CTB and MOR.

FIGS. 4A and 4B show images depicting representative REM sleep recordings in diet-induced obese (DIO) treated intranasally (IN) with vehicle and morphine or treated intranasally with leptin and morphine after a sub-lethal dose of morphine.

FIG. 4C depicts a graph showing rescuing minute ventilation (V_{E}) in diet-induced obese (DIO) treated intranasally with vehicle and morphine (M+V) or treated intranasally with leptin and morphine (M+L) after a sub-lethal dose of morphine.

FIG. 5A shows an image depicting the firing frequency of LepR^{b}+ neurons in the dorsomedial hypothalamus (DMH) that was harvested from a lean LepR^{b}−ChR2 mouse after the LepR^{b}+ neurons were treated ex vivo with vehicle or leptin.

FIG. 5B shows an image depicting HGNs (red) in the XII nucleus surrounded by LepR^{b}+ ChR2 fibers harvested from a lean LepR^{b}−ChR2 mouse.

FIG. 5C shows an image depicting increased HGN firing after photostimulation of the sournding LepR^{b}+ChR2 fibers harvested from a lean LepR^{b}−ChR2 mouse.

FIGS. 5D and 5E show images depicting robust excitatory post-synaptic currents in HGNs following photoexcitation of ChR2 expressing fibers (arrows) from LepR+ neurons harvested from a lean LepR^{b}−ChR2 mouse with no ex vivo treatment (FIG. 5D) and after application of NMDA and non-NMDA receptor antagonists AP5 and CNQX (FIG. 5E).

FIGS. 6A-6F show images depicting HGN firing with each fictive inspiratory burst recorded from the XII nerve in a “bursting slice” ex-vivo preparation from lean LepR^{b}−ChR2 mice. FIG. 6A: Shows one recording of HGN firing from an ex-vivo preparation with treatment. FIG. 6D: Shows the average recording of HGN firing from three ex-vivo preparations with no treatment. FIG. 6B: Shows one recording of HGN firing from an ex-vivo preparation following DAMGO treatment. FIG. 6E: Shows the average recording of HGN firing from three ex-vivo preparations following DAMGO treatment. FIG. 6C: Shows one recording of HGN firing from an ex-vivo preparation following DAMGO+ leptin treatment. FIG. 6F: Shows the average recording of HGN firing from three ex-vivo preparations following DAMGO+ leptin treatment.

FIG. 6G shows a graph depicting the frequency of action potential in the HGN per burst in recorded from the XII nerve in a “bursting slice” ex-vivo preparation from lean LepR^{b}−ChR2 mice in response to no treatment, DAMGO, and DAMGO+ leptin.

FIG. 7 shows a graph depicting the spontaneous excitatory post-synaptic currents recorded over time in the HGN in a “bursting slice” ex-vivo preparation from lean LepR^{b}−ChR2 mice in response to no treatment, followed by the addition of DAMGO, and the addition of leptin to the DAMGO treatment.

FIGS. 8A-8C show graphs depicting the amount of leptin in the olfactory bulbs (FIG. 8A) hypothalami (FIG. 8B), and medullae (FIG. 8C) of lean C57BL/6J mice 20 minutes after administration of either IN leptin (0.8 mg/kg, n=5) or IN vehicle. *, p<0.05. N=5.

FIGS. 9A-9E depicts graphs of respiratory parameters during non-rapid eye movement (NREM) sleep. FIG. 9A shows the frequency of inspiratory flow limited (IFL) breaths. FIG. 9B shows minute ventilation (V_{E}). FIG. 9C shows maximal inspiratory flow (V_{Imax}) during non-flow limited breaths. FIG. 9D shows severity of upper airway obstruction measured by VE during obstructed breathing. FIG. 9E shows severity of upper airway obstruction measured by V_{Imax} during obstructed breathing. FIGS. 9A-9E show graphs where Means±SEM; * represents a significant difference (p<0.05) from B; § represents a Significant difference (p<0.05) from M+L; B represents Baseline; M+V represents Morphine+IN Vehicle; M+represents Morphine+IN Leptin. All comparisons were analyzed with General Linear Model (GLM).

FIGS. 10A-10C depict images of representative recordings of apneas during non-rapid eye movement (NREM) sleep after treatment with morphine at 10 mg/kg and intranasal (IN) vehicle [Electroencephalogram (EEG), nuchal electromyogram (EMG; arbitrary units [a.u.]), respiratory flow and effort were measured continuously in freely moving mice. Bars show MEAN±Standard Error. ‡ and ‡‡ denote p<0.05 and p<0.01 respectively. All comparisons were analyzed with General Linear Model (GLM).] FIG. 10A shows an image of a recording of an obstructive apnea; upper airway obstruction was identified by continuous respiratory effort (⬆) in the presence of apnea. FIG. 10B shows an image of a recording of a central apnea identified by the absence of respiratory effort. FIG. 10C shows graph showing that leptin decreased the number of apneas per hour (n=9).

FIGS. 11A-11G depict images of representative recordings of apneas during non-rapid eye movement (NREM) in diet-induced obese (DIO) mice. FIGS. 11A-11C show representative recordings during non-rapid eye movement (NREM) sleep at baseline (B), during morphine+vehicle (M+V) and morphine+leptin (M+L) treatments. Electroencephalogram (EEG), nuchal electromyogram (EMG; arbitrary units [a.u.]), respiratory flow and effort were measured continuously in freely moving mice from 11 am to 5 pm where FIG. 11A shows the baseline recordings. FIG. 11B shows a recording depicting a severe inspiratory flow limitation (IFL) characterized by a plateau during early inspiration (*) after treatment with morphine and intranasal (IN) vehicle. FIG. 11C shows a recording depicting residual IFL (†) remaining after IN leptin in a mouse treated with morphine. FIGS. 11D-11G depict graphs of respiratory parameters during non-rapid eye movement (NREM) sleep where each line corresponds to individual data for one mouse. [Bars show Mean and SEM. ‡ and ‡‡ denote p<0.05, p<0.01 and p<0.001 respectively. All comparisons were analyzed with General Linear Model (GLM). B, Baseline; M+V, Morphine+IN Vehicle; M+L, Morphine+IN Leptin.] FIG. 11D is a graph showing that leptin abolished OIRD as evidenced by an increase in minute ventilation (V_{E}) (n=9). FIG. 11E is a graph showing that leptin decreased the frequency of obstructed (IFL) breaths (n=8). FIG. 11F is a graph showing that leptin decreased the severity of upper airway obstruction evidenced by reversals of morphine-induced reductions in maximal inspiratory flow (V_{Imax}) (n=5) during obstructed (IFL) breathing (n=5). FIG. 11G is a graph showing that leptin decreased the severity of upper airway obstruction evidenced by reversals of morphine-induced reductions in V_{E} during obstructed (IFL) breathing (n=5).

FIGS. 12A and 12B depict graphs showing that leptin restored minute ventilation to baseline by increasing both tidal volume (V_{T}) (FIG. 12A) and respiratory rate (FIG. 12B) during first 2 hours of sleep recordings (n=9). For FIGS. 12A and 12B, each line corresponds to individual data for one mouse. [Bars show means±SEM. 4 and denote p<0.05, p<0.01 and p<0.001 respectively. All comparisons were analyzed with General Linear Model (GLM). B, Baseline; M+V, Morphine+IN Vehicle; M+L, Morphine+IN Leptin.]

FIGS. 13A-13E depict graphs showing that leptin effects were no longer significant for the analysis of full 6 hours of sleep recordings (n=9). FIGS. 13A-13E show that apneas (FIG. 13A), minute ventilation (V_{E}) (FIG. 13B), frequency of obstructed (IFL) breaths (FIG. 13C) and severity of upper airway obstruction evidenced by maximal inspiratory flow (V_{Imax}) (FIG. 13D), and V_{E} (FIG. 13E) during obstructed (IFL) breathing remained unchanged after leptin treatment compared to vehicle when the full 6 hour recordings were analyzed. In the graphs shown in FIGS. 13A-13E, each line corresponds to individual data for one mouse. [Bars show Means±SEM. ‡ and ‡‡ denote p<0.05 and p<0.01 respectively. All comparisons were analyzed with General Linear Model (GLM). B, Baseline; M+V, Morphine+IN Vehicle; M+L, Morphine+IN Leptin.]

FIG. 14 depicts a graph showing that IN leptin decreased opioid-induced mortality in C57BL/6J mice treated with intranasal leptin (0.8 mg/kg in 1% BSA, n=26) or 1% BSA (vehicle, n=25) followed by intrapertitoneal morphine at 400 mg/kg. Mice were monitored for 24 hours. p=0.044.

FIGS. 15A-15G depict images showing that leptin acts on LepR^{b}+ neurons in the NTS to stimulate breathing and relieve OSA. FIG. 15A shows an image of a mouse brain following administration of pseudorabies virus (RED) to the genioglossus of LepR^{b}−Cre-GFP mice illustrating that LepR^{b} (GREEN); was absent in the XII nucleus but abundant in NTS; FIG. 15B shows an image of a mouse brain following administration of cholera toxin B (CTB-AF555) in the GG muscle of LepR^{b}−Cre-GFP mouse illustrating that hypoglossal motoneurons (RED) wrapped into LepR^{b}+ fibers (GREEN). FIG. 15C shows an image of a mouse brain where excitatory DREADD was expressed in the NTS of LepR^{b}−Cre mice. FIGS. 15D-15G depict graphs of respiratory parameters affected by either administration of the ligand J60 compared to administration of a saline, vehicle control during REM and NREM sleep. [*p<0.05. N=11.] FIG. 15D shows increases in maximal inspiratory flow (Vimax IFL) in the presence of J60. FIG. 15E shows increases in minute ventilation (V_{E} IFL) during inspiratory flow limited breathing in the presence of J60. FIG. 15F shows increases in V_{E} during non-flow limited breathing (V_{E} NFL); in the presence of J60. FIG. 15G shows a decrease in the oxygen desaturation index (ODI4%) in the presence of J60.

FIGS. 16A-16C depict images showing that a μ-opioid receptor (MOR) agonist DAMGO reduced excitatory post-synaptic current (EPSC) frequency in hypoglossal neurons (HGNs) that was reversed by application of leptin. EPSCs were recorded in vitro from HGNs (n=7). FIG. 16A shows a representative recording at Baseline, after infusion of DAMGO and Leptin. DAMGO focally applied to HGNs via a puffer pipette inhibited spontaneous EPSCs, whereas leptin application restored the frequency of EPSCs near to pre-DAMGO values. FIG. 16B shows that EPSC frequency was significantly reduced after DAMGO infusion, and Leptin infusion restored EPSC frequency to pre-DAMGO levels. FIG. 16C shows a graph of a time course of the experiment. Control EPSC frequency was quantified before and after infusion of DAMGO, followed by co-infusion of DAMGO and Leptin where ** and *** denote p<0.01 and p<0.005, respectively. [Comparisons were analyzed with one-way ANOVA test.]

FIGS. 17A and 17B depict graphs showing that leptin augmented the effect of morphine analgesia in the tail flick test. FIG. 17A shows a time course of tail flick latency for baseline (B), morphine+vehicle (M+V) and morphine+leptin (M+L), showing means±SEM. * Significant difference (p<0.05) from B; § Significant difference (p<0.05) from M+V. FIG. 17B shows that morphine increased tail flick latency at the peak of morphine analgesia, 60 minutes after morphine administration where the values are shown for individual mice (lines) and means±SEM. ‡ and ‡‡ denote p<0.05, p<0.01 and p<0.001 respectively. All comparisons were analyzed with General Linear Model (GLM). B, Baseline; M+V, Morphine+IN Vehicle; M+L, Morphine+IN Leptin.

FIG. 18 depicts a graph showing plasma leptin levels measured 1, 3, and 6 hours after IN and IP leptin administrations, as compared to baseline, n=4, for each treatment. *p<0.05.

FIGS. 19A and 19B depicts graphs showing that subcutaneous leptin increases blood pressure (BP) in mice acting in the carotid bodies. FIG. 19A shows that leptin administration increased blood pressure in C57BL6J mice FIG. 19B shows that the leptin-mediated increase in blood pressure in C57BL6J mice was abolished by carotid body (CB) denervation. [p<0.001; N=6. MAP, mean BP.]

FIGS. 20A-20C depict graphs showing that the addition of leptin at 10-100 ng/ml activated currents concentration-dependently in LepR^{b} expressing PC12 cells. FIG. 20A shows a time-course of the concentration-dependent effect of leptin on the out-ward current elicited in a PC12 cell at +100 mV. FIG. 20B shows a representative I-V relation generated by a ramp protocol from −100 to +100 mV under control condition or in the presence of 10, 30, 100 ng/ml leptin, and leptin+FTY720 (3 μM). FIG. 20C shows a bar graph showing the concentration-dependent effect of leptin generated from 6 different cells.

FIGS. 21A and 21B depict graphs showing the effect of Allo-aca on the leptin-activated TRPM7 current in PC12 cells. FIG. 21A shows a time-course of the leptin-activated out-ward current before and after the application of 300 nM Allo-aca. FIG. 21B shows a representative tracings generated by the ramp protocol in control (a, black), leptin (b, red) and leptin+allo-aca (c, blue).

### DETAILED DESCRIPTION

The primary cause of death associated with opiates is opiate-mediated respiratory suppression (ORS). Opiates act on μ-opioid receptors (MOR) in the preBötzinger complex, the ventral respiratory group, as well as potentially other respiratory centers in the brain. Opioid action on in the respiratory center of the brain causes a decreased respiratory rate. However, ORS does not only occur from the opiate action in the respiratory center of the brain but upper airway obstruction during opiate-induced sedation is an additional mechanism of ORS and mortality induced by opiates. This places obese subjects at a high risk for death due to ORS as the prevalence of upper airway obstructions, such as obstructive sleep apnea (OSA), is much higher in these subj ects.

Mechanisms of opiate-induced upper airway obstruction are not fully understood however, as disclosed in the data herein, opioids directly suppress hypoglossal motoneuron (HGN) activity by acting on the MORs expressed on the hypoglossal motoneurons innervating a muscle that controls tongue movement, the genioglossus muscle. The suppression of genioglossus muscle activity by opioid use can result in an upper airway obstruction contributing to the probability of death associated with ORS. The present disclosure is based in part on the surprising discovery that leptin can reverse and prevent opioid action on hypoglossal motoneuron activity despite there being no receptors for leptin on hypoglossal motoneurons. This action is likely by effects on presynaptic terminals that surround and excite hypoglossal motorneurons that possess leptin receptors. Accordingly, administering leptin can treat ORS. Further, leptin can be used to treat opioid overdose in combination with naloxone or as a substitute for naloxone.

Two complicating factors toward the use of leptin for treatment of ORS is that leptin does not readily cross the blood brain barrier and oral delivery of leptin has not been effective in leptin-resistant subjects, such as obese subjects. To circumvent this problem, embodiments herein describe compositions and methods of administering leptin intranasally. In some aspects, leptin formulation for intranasal administration may be a spray, nasal drops, or a combination thereof.

The present disclosure is based in part on the surprising discovery that leptin relieved opioid-induced hypoventilation and obstructive sleep apnea and reversed the opioid-induced depression of excitatory synaptic neurotransmission to hypoglossal motor neurons (HGNs). Accordingly, the present disclosure provides methods of treating opioid overdose or opioid-mediated respiratory suppression with intranasal leptin. Another aspect of the present disclosure provides methods of treating opioid overdose or opioid-mediated respiratory suppression with the combination of intranasal leptin and intranasal anti-opioid, such as naloxone. Other aspect of the present disclosure provides compositions encompassing a combination of leptin and an anti-opioid, such as naloxone, formulated for intranasal administration.

### I. Compositions

Some embodiments of the present disclosure provide for compositions for treating opioid-induced respiratory depression in a subject in need thereof. In some embodiments, compositions disclosed herein can include leptin. In some embodiments, compositions disclosed herein can include at least one pharmaceutical excipient. In some embodiments, compositions disclosed herein can be formulated for intranasal administration.

A. Leptin and Leptin Variants

In various embodiments, compositions disclosed herein can include leptin or a variant thereof. Leptin is a protein product of the obese gene (ob), and can be found in several different mammalian species, including mice, humans, pigs, and cattle. Human endogenous leptin, in its mature form, is a 146-amino acid protein (See GenBank Accession number BAA09787). The full length amino acid sequence of human leptin is:

(SEQ ID NO: 1)
MHWGTLCGFLWLWPYLFYVQAVPIQKVQDDTKTLIKTIVTRINDISHTQS
VSSKQKVTGLDFIPGLHPILTLSKMDQTLAVYQQILTSMPSRNVIQISND
LENLRDLLHVLAFSKSCHLPWASGLETLDSLGGVLEASGYSTEVVALSRL
QGSLQDMLWQLDLSPGC

In various embodiments, compositions disclosed herein can include a recombinant leptin. In some aspects, a recombinant leptin can be a recombinant human leptin. In some aspects, a recombinant leptin can be modified. Modification of a recombinant leptin can introduce one or more changes to the native amino acid sequence, addition of at least one conjugate, or a combination thereof. In some examples, at least one conjugate can be added to a recombinant leptin. In some examples, the conjugate can be a polymer. Non limiting examples of polymers can include poly(ethylene glycol) (PEG), N-(2-hydroxypropyl)methacrylamide (HPMA), poly(vinylpyrrolidone) (PVP), poly(n-acryloylmorpholine) (pNAcM), poly [(ethylene oxide)-co-(methylene ethylene oxide)], hydroxyethyl starch (HES), polysialic acid (PSA), dextrin, poly(glutamic acid) (PGA), poly(carboxybetaine) (PCB), poly(2-oxazoline) (POZ), poly(N-hydroxypropyl)methacyrlamide) (pHPMA), poly(poly(ethylene glycol) methyl ether methacrylate) (pPEGMA), PG, poly(glycerol) (PG), polyglutamic acid (PGA), or a combination thereof. In some examples, a recombinant leptin can be a pegylated recombinant human leptin (PEG-OB).

In some embodiments, a leptin included in compositions described herein can be a leptin polypeptide or a variant of a leptin polypeptide. In some aspects, a variant of a leptin polypeptide may bind to and activate the leptin receptor. In some examples, a variant of a leptin polypeptide can be a leptin polypeptide with conservative amino acid substitutions, whereby amino acids are substituted with alternative amino acids of similar stereochemistry, i.e., charge or hydrophobicity. In some aspects, a leptin polypeptide disclosed herein may be a variant resulting from alternative post-translational modification, including glycosylation, acylation, methylation, phosphorylation, sulfation, or proteolytic cleavage. In other aspects, a leptin polypeptide disclosed herein may be a polypeptide with amino acid sequences which are about 95%, 90%, 85%, 80%, 75%, or about 70% identical to the human leptin sequence (SEQ ID NO: 1), as calculated by algorithms known in the art, for example BLAST, FASTA, or Smith-Waterman. In some examples, a leptin polypeptide disclosed herein can be a recombinant human methionyl leptin.

In some embodiments, a leptin polypeptide disclosed herein can be a biologically active fragment of leptin. In some aspects, leptin polypeptides may comprise fragments of the leptin polypeptides above; or a polypeptide comprising any one or more of the polypeptides above or a functional derivative, analogue or variant thereof In some examples, a biologically active fragment of leptin can be a leptin polypeptides having at least 4 amino acids. In some examples, a biologically active fragment of leptin can be a leptin polypeptides having about 4 amino acids to about 50 amino acids. In some aspects, a biologically active fragment of leptin can have about 95%, 90%, 85%, 80%, 75%, or about 70% identical to the human leptin sequence (SEQ ID NO: 1). The biologically active fragments of leptin of the present disclosure may be prepared using conventional digestion methods, synthetic techniques or by use of standard expression methodology.

In some embodiments, a leptin included in compositions described herein can optionally include an Fc immunoglobulin domain. In some embodiments, the leptin is a humanized leptin optimally including at least a portion of an immunoglobulin constant region or domain (Fc), typically that of a human immunoglobulin. Leptins disclosed herein may have Fc regions modified as described in WO 99/58572. Humanized leptins may also involve affinity maturation. Methods for constructing humanized proteins are also well known in the art. See, e.g., Rath et al., Crit Rev Biotechnol. 35(2):235-254 (2015).

In various embodiments, a leptin included in compositions described herein can optionally include a human Fc domain fusion partner. In some embodiments, the human Fc domain fusion partner comprises the entire Fc domain. In some embodiments, a fusion peptide of leptin and an Fc fragment of immunoglobulin can encompass one or more fragments of the Fc domain. For example, the fusion peptide may include a hinge and the CH2 and CH3 constant domains of a human IgG, for example, human IgG1, IgG2, or IgG4. In some embodiments, fusion peptide disclosed herein can encompass a variant Fc polypeptide or a fragment of a variant Fc polypeptide. The variant Fc may comprise a hinge, CH2, and CH3 domains of human IgG. In an embodiment, a fusion peptide herein may be a homodimeric protein linked through at least one residue in the hinge region of an IgG Fc. An exemplary human Fc domain is:

(SEQ ID NO: 2)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK

In some embodiments, the decoy fusion proteins disclosed herein may comprise a Fc domain that is at least 80% (e.g., 85%, 90%, 95%, or 98%) sequence identity, individually or collectively, as compared with the SEQ ID NO: 2. Methods of making fusion peptides are well-known in the art. In some embodiments, a Fc domain can be linked to the N-terminus of a leptin protein (e.g., leptin or a biologically-active fragment of leptin) or, alternatively, the leptin protein can be linked to the N-terminus of the Fc domain. The Fc domain may comprise a linker, for example, a peptide linker, which may or may not comprise an enzyme cleavage site. A peptide linker may include at least 1 amino acid residue (natural or non-natural) between the Fc domain and leptin protein. In some embodiments, a Fc domain and a leptin protein are attached by an amino acid linker that is about 1 to about 10 amino acids in length. Alternatively, or in addition, other peptide and non-peptide linkers may also be used to link one or more of the Fc domains and leptin proteins disclosed herein. In some embodiments, Fc domains herein may also comprise a molecule that extends the in vivo half-life by imparting improved receptor binding to the leptin protein within an acidic intracellular compartment, for example, an acid endosome or a lysosome.

In some embodiments, a fusion leptin protein may optionally include a signal peptide. A signal peptide can enhance specificity of binding to a target protein, be used leptin protein generation and purification in culture medium. Signal peptides can be derived from antibodies, such as, but not limited to, CD8 or CD4, as well as epitope tags such as, but not limited to, GST or FLAG. In some embodiments, a signal sequence be located C-terminally of the leptin protein. Other signal peptides may be used. In other embodiments, fusion leptin protein may optionally include a cleavage site between a signal peptide and the C-terminus of the leptin protein.

In some embodiments, a leptin included in compositions described herein can be a leptin peptidomimetic. As used herein, the terms “leptin mimic, leptin mimetic or leptin peptidomimetic” are used interchangeably herein to refer to a leptin derivative comprising a functional domain of the leptin protein, alone or in combination with another molecule, which will produce a biological effect. As an example, a peptidomimetic is a compound containing non-peptidic structural elements capable of mimicking or antagonizing (meaning neutralizing or counteracting) the biological action(s) of a natural parent peptide. In some examples, a leptin disclosed herein can be a peptidomimetic incorporating the portion of leptin mediating activity that is of a size small enough to penetrate the blood-brain barrier.

In various embodiments, compositions disclosed herein can include both polypeptide and non-polypeptide compounds that activate the leptin receptor. In some embodiments, compounds that activate the leptin receptor may also encompass functional derivatives of leptin polypeptides, including salts and solvates of the polypeptides mentioned herein. Additionally, the leptin polypeptides may be chemically modified by the attachment of groups or moieties so as to improve the physical properties, such as stability, or the therapeutic properties, for example the pharmacokinetic properties, of the polypeptide.

In some embodiments, a compound that activates the leptin receptor is a non-polypeptide agonist or a small molecule agonist. In some aspects, a compound that activates a leptin receptor may be an antibody. In some examples, an antibody could bind to and activate the leptin receptor such that the JAK/STAT, AMPK, and/or P13 kinase signaling pathways are activated. In some embodiments, a leptin included in compositions described herein can be a leptin agonist. A leptin agonist is a compound, small molecule, or polypeptide capable of activating the leptin receptor and/or downstream effectors. In some embodiments, a leptin agonist can target one or more downstream efforts of leptin. In some examples, an activator of AMP-dependent protein kinase (AMPK) may be a leptin agonist. Non-limiting examples of AMPK activators include phenformin, 5-aminoimidazole-4-carboxamide riboside (AICAR), metformin and rosiglitazone.

i. Methods of Making Leptin and Leptin Variants

Any of the leptin proteins and leptin protein variants disclosed herein can be made by any method known in the art. If desired, a leptin protein of interest may be sequenced and the polynucleotide sequence may then be cloned into a vector for expression or propagation. The sequence encoding the leptin protein of interest may be maintained in vector in a host cell and the host cell can then be expanded and frozen for future use. In an alternative, the polynucleotide sequence may be used for genetic manipulation to, e.g., humanize the leptin protein or to improve the affinity (affinity maturation), or other characteristics of the leptin protein. For example, where a leptin protein-is fused to a Fc fragment, the Fc region may be engineered to more resemble human Fc regions to avoid immune response if the leptin protein is from a non-human source and is to be used in clinical trials and treatments in humans. Alternatively or in addition, it may be desirable to genetically manipulate the leptin sequence to obtain greater affinity and/or specificity to the target protein and greater efficacy in binding. It will be apparent to one of skill in the art that one or more polynucleotide changes can be made to the leptin protein and still maintain its binding specificity to the target protein.

Genetically engineered leptin proteins, such as recombinant human leptin, recombinant human methionyl leptin, leptin peptidomimetic, biologically active fragments of leptin, fusion peptides of leptin with an Fc fragment of immunoglobulin, and fusion peptides of the biologically-active fragment of leptin with the Fc fragment of immunoglobulin, can be produced via, e.g., conventional recombinant technology. In one example, DNA encoding a leptin proteins specific to a target protein can be readily isolated and sequenced using conventional procedures (e.g., by using oligonucleotide probes that are capable of binding specifically to genes encoding the Fc and decoy protein fragment). Once isolated, the DNA may be placed into one or more expression vectors, which are then transfected into host cells such as E. coli cells, simian COS cells, Chinese hamster ovary (CHO) cells, Human Embryotic Kindey (HEK) 293 cells or myeloma cells that do not otherwise produce the decoy fusion proteins herein. The DNA can then be modified, for example, by substituting the coding sequence for human Fc domains in place of the homologous murine sequences, Morrison et al., (1984) Proc. Nat. Acad. Sci. 81:6851, or by covalently joining to the Fc coding sequence all or part of the coding sequence for a non-immunoglobulin polypeptide.

In some examples, leptin proteins disclosed herein are prepared by recombinant technology as exemplified below. Generally, a nucleic acid sequence encoding one or all proteins included in a leptin protein disclosed herein can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the leptin proteins.

A variety of promoters can be used for expression of the leptin proteins described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the leptin proteins herein may be introduced into suitable host cells for producing the leptin proteins. The host cells can be cultured under suitable conditions for expression of the leptin protein or any polypeptide chain thereof. Such leptin proteins or polypeptide chains thereof can be recovered by the cultured cells (e.g., from the cells or the culture supernatant) via a conventional method, e.g., affinity purification. If necessary, leptin proteins can be incubated under suitable conditions for a suitable period of time allowing for production of the decoy fusion protein.

In some embodiments, methods for preparing a leptin protein described herein involve a recombinant expression vector that encodes all components of the leptin proteins as also described herein. The recombinant expression vector can be introduced into a suitable host cell (e.g., a HEK293T cell or a dhfr-CHO cell) by a conventional method, e.g., calcium phosphate-mediated transfection. Positive transformant host cells can be selected and cultured under suitable conditions allowing for the expression of the leptin proteins which can be recovered from the cells or from the culture medium.

Standard molecular biology techniques are used to prepare the recombinant expression vector, transfect the host cells, select for transformants, culture the host cells and recovery of the leptin proteins from the culture medium. For example, some leptin proteins can be isolated by affinity chromatography with a Protein A or Protein G coupled matrix. In some examples, leptin proteins herein may include a tag and the like to isolate and/or purify the decoy fusion protein. In other examples, leptin proteins herein may be subjected to enzymatic cleavage to remove a tag, linker, signaling peptide, or a combination thereof after purification.

Any of the nucleic acids encoding the leptin proteins as described herein, vectors (e.g., expression vectors) containing such; and host cells comprising the vectors are within the scope of the present disclosure.

B. Naloxone, Naloxone Related Substances, and Pharmaceutically Acceptable Salts

In certain embodiments, compositions disclosed herein can further include, but are not limited to, naloxone, a naloxone related substance, or a pharmaceutically acceptable salt thereof. Naloxone is a non-selective and competitive opioid receptor antagonist. It is a synthetic morphinan derivative derived from oxymorphone (14-hydroxydihydromorphinone), an opioid analgesic. Naloxone is also known as N-allylnoroxymorphone or as 17-allyl-4,5α-epoxy-3,14-dihydroxymorphinan-6-one. As used herein, “naloxone related substances” can refer to a compound selected from the following: 10-α-hydroxynaloxone, oxymorphone, noroxymorphone, 10-β-hydroxynaloxone, 7,8-didehydronaloxone, 2,2′-bisnaloxone, and 3-O-allynlnaloxone.

In some embodiments, naloxone and/or a naloxone related substance used in the compositions herein can be in the form of an ester prodrug. The term “ester” herein can refer a compound which is produced by modifying a functional group (e.g. hydroxyl, carboxyl, amino or the like group). Examples of the “ester” include “esters formed with a hydroxyl group” and “esters formed with a carboxyl group”. The term “ester” means an ester whose ester residue is a “conventional protecting group” or a “protecting group removable in vivo by a biological method such as hydrolysis”. In some embodiments, the term “conventional protecting group” can mean a protecting group removable by a chemical method such as hydrogenolysis, hydrolysis, electrolysis or photolysis. In other embodiments, the term “protecting group removable in vivo by a biological method such as hydrolysis” can mean a protecting group removable in vivo by a biological method such as hydrolysis to produce a free acid or its salt.

In other embodiments, naloxone and/or a naloxone related substance used in the compositions herein can be in the form of a pharmaceutically acceptable salt. By “salt” or “pharmaceutically acceptable salt”, it is meant those salts which are, within the scope of sound medical judgment, suitable for use in contact with the tissues of humans and lower animals without undue toxicity, irritation, and allergic response, commensurate with a reasonable benefit to risk ratio, and effective for their intended use. A “pharmacologically acceptable salt” means a salt, which can be formed when naloxone and/or a naloxone related substance has an acidic group such as carboxyl or a basic group such as amino or imino. In some examples, a naloxone and/or a naloxone related substance salt formed with an acidic group can include alkali metal salts such as a sodium salt, potassium salt or lithium salt, alkaline earth metal salts such as a calcium salt or magnesium salt, metal salts such as an aluminum salt or iron salt; amine salts, e.g., inorganic salts such as an ammonium salt and organic salts such as a t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt or tris(hydroxymethyl)aminomethane salt; and amino acid salts such as a glycine salt, lysine salt, arginine salt, ornithine salt, glutamate or aspartate. In other embodiments, a leptin salt formed with a basic group can include hydro-halides such as a hydrofluoride, hydrochloride, hydrobromide or hydroiodide, inorganic acid salts such as a nitrate, perchlorate, sulfate or phosphate; lower alkanesulfonates such as a methanesulfonate, trifluoromethanesulfonate or ethanesulfonate, arylsulfonates such as a benzenesulfonate or p-toluenesulfonate, organic acid salts such as an acetate, malate, fumarate, succinate, citrate, ascorbate, tartrate, oxalate or maleate; and amino acid salts such as a glycine salt, lysine salt, arginine salt, ornithine salt, glutamate or aspartate. In certain embodiments, when a pharmacologically acceptable salt of naloxone and/or a naloxone related substance is allowed to stand in the atmosphere or is recrystallized, it can absorb water to form a hydrate.

In other embodiments, naloxone and/or a naloxone related substance used in the compositions disclosed herein can be in the form of another naloxone and/or a naloxone related substance derivative. In some examples, the term “other derivative” can mean a derivative of the above naloxone and/or a naloxone related substance other than the above-described “ester” or the above-described “pharmacologically acceptable salt” which can be formed, if it has an amino and/or carboxyl group or other conjugate form.

C. Nasal Formulations

In certain embodiments, nasal formulations can include, but are not limited to, a leptin or a variant thereof as disclosed herein. In some embodiments, nasal formulations can further include naloxone, a naloxone related substance, or a pharmaceutically acceptable salt thereof

In some embodiments, nasal formulations disclosed herein can include leptin in the form of a particle. In some aspects, the particle form of leptin is a solid particle. In other embodiments, the particle form of leptin is a semi-solid particle. In yet other embodiments, a particle of leptin can be prepared by those of skill in the art using known methods for such preparation. Non-limiting examples of methods of preparing leptin particles include spray-drying, evaporation, micronization, nanosization, and crystallization or other known methods.

In some embodiments, nasal formulations can have leptin in the form of a particle, the particle having a particle size less than 10, 9.5, 9.0, 8.5, 8.0, 7.5, 7.0, 6.5, 6.0, 5.5, 5.0, 4.5, or 0.40 microns. In embodiments, nasal formulations can have leptin in the form of a particle where greater than 90% or about 100% of the leptin particles have a particle size less than 15 microns. In some embodiments, particles containing leptin can be part of an enhanced formulation for nasal delivery and mucosal adhesion.

In some embodiments, nasal formulations disclosed herein can have about 0.01 mg/ml to about 20 mg/ml leptin. In some aspects, nasal formulations disclosed herein can have about 0.01 mg/ml, about 0.1 mg/ml, about 0.5 mg/ml, about 1.0 mg/ ml, about 2.5 mg/ml, about 5 mg/ml, about 7.5 mg/ml, about 10 mg/ml or about 20 mg/ml leptin.

In some embodiments, nasal formulations disclosed herein can be a solution, a suspension or an emulsion. In some embodiments, nasal formulations disclosed herein can have least one pharmaceutically acceptable carrier or diluent. In some aspects, pharmaceutically acceptable carriers and diluents suitable for use herein can be selected from solid carriers or diluents, liquid carriers or diluents, gel carriers or diluents or a combination thereof.

In certain embodiments, nasal formulations disclosed herein can be a solution, a suspension or an emulsion. In accordance with these embodiments, nasal formulations disclosed herein can include, but are not limited to, polymers of carbopol, chitosan, sodium carboxymethyl cellulose (NaCMC), hydroxypropyl methylcellulose (HPMC), hydroxypropyl cellulose methylcellulose, poloxamer, polyoxyethylene, pluronic-poly(acrylic acid) copolymer, carbomer, chitosan, polyvinyl alcohol (PVA), poly(N-isopropylacrylamide) (PNiPAAm), methocel A4M, polymethacrylic acid and polyethylene glycol (P(MAA-g-EG), polyvinylacetal diethylamino acetate, or a combination thereof.

In certain embodiments, a carrier or diluent can be a liquid carrier or diluent comprising at least one of water, propylene glycol and pharmaceutically acceptable alcohols. Pharmacologically suitable fluids for use herein include, but are not limited to, polar solvents, including, but not limited to, compounds that contain hydroxyl groups or other polar groups. Solvents include, but are not limited to, water or alcohols, such as ethanol, isopropanol, and glycols including propylene glycol, polyethylene glycol, polypropylene glycol, glycol ether, glycerol and polyoxyethylene alcohols. Polar solvents also include protic solvents, including, but not limited to, water, aqueous saline solutions with one or more pharmaceutically acceptable salt(s), alcohols, glycols or a mixture thereof. In one alternative embodiment, the water for use in the present formulations should meet or exceed the applicable regulatory requirements for use in inhaled drugs.

In some embodiments, a carrier or diluent can be a gel carrier or diluent or a combination thereof In other embodiments, the leptin can be a viscous liquid. In certain embodiments, nasal formulations herein can include at least one viscosity and/or density enhancing agent. Examples of viscosity and/or density enhancing agents that can be added include carboxymethylcellulose (CMC), veegum, tragacanth, bentonite, hydroxypropylmethylcellulose, hydroxypropylcellulose, hydroxyethylcellulose, poloxamers (e.g. poloxamer 407), polyethylene glycols, alginates xanthym gums, carageenans and carbopols. In other embodiments, a viscosity enhancing agent for use herein can possess thixotropic properties which ensure that the formulation assumes a gel-like appearance at rest, characterized by a high viscosity value. In certain methods, if a nasal formulation disclosed herein is subjected to shear forces, such as those caused by agitation prior to spraying, the viscosity of the formulation can decrease transiently to such a level to enable it to flow readily through the spray device and exit as a fine mist spray. In some embodiments, a mist as disclosed herein can be capable of infiltrating the mucosal surfaces of the anterior regions of the nose (frontal nasal cavities), the frontal sinus, the maxillary sinuses and the turbinate which overlies the conchas of the nasal cavities. Once deposited in a subject, the viscosity of the nasal formulations disclosed herein can increase to a sufficient level to assume a gel-like form and remain in the nasal mucosa longer for improved treatment. In certain embodiments, nasal formulations herein can include a viscosity enhancing agent in an amount of about 0.1% (w/w) to about 5% (w/w), based on the total weight of the formulation.

In some embodiments, the disclosed nasal formulations can include at least one adherence agent for prolonging nasal mucosal interaction of the formulation. In some examples, an adherence agent for use herein can be a cellulose or a derivative thereof, a starch, a wax, a gel, a synthetic polymer, a natural polymer, and the like. In some embodiments, the disclosed nasal formulations can include at least one polymer. In some examples, a polymer suitable for use in the formulations herein can be a mucoadhesive polymer. A “mucoadhesive polymers” as understood herein is a natural or synthetic macromolecules capable of adhering to mucosal tissue surfaces. In some aspects, a polymer suitable for use in can be carbopol, chitosan, sodium carboxymethyl cellulose (NaCMC), hydroxypropyl methylcellulose (HPMC), hydroxypropyl cellulose methylcellulose, poloxamer, polyoxyethylene, pluronic-poly(acrylic acid) copolymer, carbomer, chitosan, polyvinyl alcohol (PVA), poly(N-isopropylacrylamide) (PNiPAAm), methocel A4M, polymethacrylic acid and polyethylene glycol (P(MAA-g-EG), polyvinylacetal diethylamino acetate, or a combination thereof In some examples, nasal formulations herein can include a mucoadhesive polymer in an amount of between about 0.1% (w/w) to about 25% (w/w), based on the total weight of the formulation.

In some embodiments, the disclosed nasal formulations can have a pH of about 2.0 to about 9.0. Optionally, the nasal formulations herein can have a pH buffer. Such a buffer can include any known pharmaceutically suitable buffers which are physiologically acceptable upon administration intranasally.

In some embodiments, nasal formulations disclosed herein can be free of pathogenic organisms (e.g., sterile). In some embodiments, the nasal compositions are formulated to be a pharmaceutical formulation. Processes which can be considered for achieving sterility include any appropriate sterilization steps known in the art. In some embodiments, leptin can be produced under sterile conditions, and the mixing and packaging is conducted under sterile conditions. In other embodiments, the nasal formulations disclosed herein can be sterile filtered and filled in vials, including unit dose vials providing sterile unit dose formulations which are used in a nasal spray device for example. Each unit dose vial can be sterile and can be suitably administered without contaminating other vials or the next dose. In some aspects, one or more ingredients in the nasal formulations herein can be sterilized by steam, gamma radiation or prepared using or mixing sterile steroidal powder and other sterile ingredients where appropriate. In other aspects, nasal formulations here can be prepared and handled under sterile conditions, or can be sterilized before or after packaging.

In addition to or in lieu of sterilization, nasal formulations disclosed herein can include a pharmaceutically acceptable preservative. Preservatives suitable for use herein include, but are not limited to, those that protect the solution from contamination with pathogenic particles, including phenylethyl alcohol, benzalkonium chloride or benzoic acid, or benzoates such as sodium benzoate and phenylethyl alcohol. In some examples, the preservative for use in the present formulations is benzalkonium chloride. In certain embodiments, the formulations herein comprise from about 0.001% to about 10.0% w/w of benzalkonium chloride, or from about 0.01% v/w phenylethyl alcohol. Preserving agents can also be present in an amount from about 0.001% to about 1% w/w.

In some embodiments, nasal formulations provided herein can include from about 0.001% to about 90%, or about 0.001% to about 50%, or about 0.001% to about 25%, or about 0.001% to about 10%, or about 0.001% to about 1% of one or more emulsifying agent, wetting agent, or suspending agent. Such agents for use herein include, but are not limited to, polyoxyethylene sorbitan fatty esters or polysorbates, including, but not limited to, polyethylene sorbitan monooleate (Polysorbate 80), polysorbate 20 (polyoxyethylene (20) sorbitan monolaurate), polysorbate 65 (polyoxyethylene (20) sorbitan tristearate), polyoxyethylene (20) sorbitan mono-oleate, polyoxyethylene (20) sorbitan monopalmitate, polyoxyethylene (20) sorbitan monostearate; lecithins; alginic acid; sodium alginate; potassium alginate; ammonium alginate; calcium alginate; propane-1,2-diol alginate; agar; carrageenan; locust bean gum; guar gum; tragacanth; acacia; xanthan gum; karaya gum; pectin; amidated pectin; ammonium phosphatides; microcrystalline cellulose; methylcellulose; hydroxypropylcellulose; hydroxypropylmethylcellulose; ethylmethylcellulose; carboxymethylcellulose; sodium, potassium and calcium salts of fatty acids; mono-and di-glycerides of fatty acids; acetic acid esters of mono- and di-glycerides of fatty acids; lactic acid esters of mono-and di-glycerides of fatty acids; citric acid esters of mono-and di-glycerides of fatty acids; tartaric acid esters of mono-and di-glycerides of fatty acids; mono-and diacetyltartaric acid esters of mono-and di-glycerides of fatty acids; mixed acetic and tartaric acid esters of mono-and di-glycerides of fatty acids; sucrose esters of fatty acids; sucroglycerides; polyglycerol esters of fatty acids; polyglycerol esters of polycondensed fatty acids of castor oil; propane-1,2-diol esters of fatty acids; sodium stearoyl-2lactylate; calcium stearoyl-2-lactylate; stearoyl tartrate; sorbitan monostearate; sorbitan tristearate; sorbitan monolaurate; sorbitan monooleate; sorbitan monopalmitate; extract of quillaia; polyglycerol esters of dimerised fatty acids of soya bean oil; oxidatively polymerised soya bean oil; and pectin extract. In certain embodiments herein, the present formulations comprise polysorbate 80, microcrystalline cellulose, carboxymethylcellulose sodium and/or dextrose.

In some embodiments, nasal formulations provided herein can include from about 0.001% to about 90%, or about 0.001% to about 50%, or about 0.001% to about 25%, or about 0.001% to about 10%, or about 0.001% to about 1% of one or more pharmacologically suitable excipients and additives. Excipients and additives generally have no pharmacological activity, or at least no undesirable pharmacological activity. The concentration of these can vary with the selected agent, although the presence or absence of these agents, or their concentration is not an essential feature of the invention. Excipients and additives suitable for use herein can include, but are not limited to, surfactants, moisturizers, stabilizers, complexing agents, antioxidants, or other additives known in the art. Complexing agents include, but are not limited to, ethylenediaminetetraacetic acid (EDTA) or a salt thereof, such as the disodium salt, citric acid, nitrilotriacetic acid and the salts thereof. In some aspects, nasal formulations herein can include a humectant. In some examples, nasal formulations herein can include from about 0.001% to about 5% by weight of a humectant to inhibit drying of the mucous membrane and to prevent irritation. Any of a variety of pharmaceutically-acceptable humectants can be employed, including, but not limited to, sorbitol, propylene glycol, polyethylene glycol, glycerol or mixtures thereof, and the like.

In some embodiments, nasal formulations provided herein can include one or more solvents or co-solvents to increase the solubility of any of the components of the present formulation. Solvents in the formulations described herein can be about 0.001% to about 90%, or about 0.001% to about 50%, or about 0.001% to about 25%, or about 0.001% to about 10%, or about 0.001% to about 10% of one or more solvents or co-solvents. Solvents or co-solvents for use herein include, but are not limited to, hydroxylated solvents or other pharmaceutically-acceptable polar solvents, such as alcohols including isopropyl alcohol, glycols such as propylene glycol, polyethylene glycol, polypropylene glycol, glycol ether, glycerol, and polyoxyethylene alcohols.

In some embodiments, nasal formulations provided herein can include at least one tonicity agent. Tonicity agents for use herein can include, but are not limited to sodium chloride, potassium chloride, zinc chloride, calcium chloride or mixtures thereof. Other osmotic adjusting agents can also include, but are not limited to, mannitol, glycerol, and dextrose or mixtures thereof. In some examples, the nasal formulations herein can have about 0.01% to about 8% w/w, or 1% to about 6% w/w total amount of tonicity agent(s).

In some embodiments, nasal formulations provided herein can be stable. As used herein, the stability of formulations provided herein refers to the length of time at a given temperature that greater than 80%, 85%, 90% or 95% of the initial amount of drug substance, (e.g., leptin) is present in the formulation. For example, but not limited to, the nasal formulations provided herein can be stored between about 15° C. and about 30° C., and remain stable for at least 1, 2, 12, 18, 24 or 36 months. Also, the nasal formulations can be suitable for administration to a subject in need thereof after storage for more than 1, 2, 12, 18, 24 or 36 months at 25° C. In some examples, more than 80%, or more than 85%, or more than 90%, or more than 95% of the initial amount of drug substance (e.g., leptin) remains after storage of the formulations for more than 1, 2, 12, 18, 24 or 36 months between about 15° C. and about 30° C.

The nasal formulations of the present disclosure can be manufactured in any conventional manner. In some examples, nasal formulations herein can be made by thoroughly mixing the ingredients described herein at ambient or elevated temperatures in order to achieve solubility of ingredients where appropriate. In some aspects, the preparation of leptin having the particle size distribution profile of the present invention can be obtained by any conventional means known in the art, or by minor modification of such means. For example, suspensions of leptin particles can rapidly undergo particulate size reduction when subjected to “jet milling” (high pressure particle in liquid milling) techniques. Other known methods for reducing particle size into the micrometer range include mechanical milling, the application of ultrasonic energy and other techniques.

In some embodiments, nasal formulations disclosed herein can incorporate lipid or fatty acid based carriers, processing agents, or delivery vehicles, to provide improved formulations for mucosal delivery of leptin. For example, a variety of formulations and methods are provided for mucosal delivery which include leptin, admixed or encapsulated by, or coordinately administered with, a liposome, mixed micellar carrier, or emulsion, to enhance chemical and physical stability and increase the half life of the drug (e.g., by reducing susceptibility to proteolysis, chemical modification and/or denaturation) upon mucosal delivery. Like liposomes, unsaturated long chain fatty acids, which also have enhancing activity for mucosal absorption, can form closed vesicles with bilayer-like structures (so-called “ufasomes”). These can be formed, for example, using oleic acid to entrap biologically active peptides and proteins for mucosal, e.g., intranasal, delivery within this disclosure. Other delivery systems within this disclosure can combine the use of polymers and liposomes to ally the advantageous properties of both vehicles such as encapsulation inside the natural polymer fibrin.

In some embodiments, nasal formulations can include long and medium chain fatty acids, as well as surfactant mixed micelles with fatty acids. Most naturally occurring lipids in the form of esters have important implications with regard to their own transport across mucosal surfaces. Free fatty acids and their monoglycerides which have polar groups attached have been demonstrated in the form of mixed micelles to act on the intestinal barrier as penetration enhancers. This discovery of barrier modifying function of free fatty acids (carboxylic acids with a chain length varying from 12 to 20 carbon atoms) and their polar derivatives has stimulated extensive research on the application of these agents as mucosal absorption enhancers. In some examples, nasal formulations herein can include long chain fatty acids, especially fusogenic lipids (unsaturated fatty acids and monoglycerides such as oleic acid, linoleic acid, linoleic acid, monoolein, etc.) provide useful carriers to enhance mucosal delivery of insulin, analogs and mimetics, and other biologically active agents disclosed herein. Medium chain fatty acids (C6 to C12) and monoglycerides have also been shown to have enhancing activity in intestinal drug absorption and can be adapted for use within the mucosal delivery formulations and methods of this disclosure. In addition, sodium salts of medium and long chain fatty acids are effective delivery vehicles and absorption-enhancing agents for mucosal delivery of biologically active agents within this disclosure. Fatty acids can be employed in soluble forms of sodium salts or by the addition of non-toxic surfactants, e.g., polyoxyethylated hydrogenated castor oil, sodium taurocholate, etc. Other fatty acid and mixed micellar preparations that are within this disclosure include, but are not limited to, Na caprylate (C8), Na caprate (C10), Na laurate (C12) or Na oleate (C18), optionally combined with bile salts, such as glycocholate and taurocholate.

In some embodiments, nasal formulations of the present disclosure can be formulated into a dosage form for pharmaceutical administration. Suitable dosage forms include, without limit, liquids, ointments, creams, emulsions, lotions, gels, bioadhesive gels, sprays, aerosols, pastes, foams, sunscreens, capsules, microcapsules, suspensions, pessary, powder, semi-solid dosage form, etc. In some embodiments, the formulations presented herein can be formulated into a liquid dispersion, gel, aerosol, nasal aerosol, ointment, cream, semi-solid, or suspension. In some embodiments, nasal formulations according to the present disclosure can be a drop delivery formulation. In some embodiments, nasal formulations according to the present disclosure can be a spray or atomizer delivery formulation.

Pharmaceutically acceptable compositions of this disclosure can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

Pharmaceutical compositions for inhalation or insufflation include solutions and suspensions in pharmaceutically acceptable, aqueous or organic solvents, or mixtures thereof, and powders. The liquid or solid compositions can contain suitable pharmaceutically acceptable excipients as set out above. In some embodiments, the compositions are administered by the oral or nasal respiratory route for local or systemic effect.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In the case of a pressurized aerosol, the dosage unit can be determined by providing a valve to deliver a metered amount. The pressurized container, pump, spray or nebulizer can contain a solution or suspension of the active compound, e.g. using a mixture of ethanol and the propellant as the solvent, which can additionally contain a lubricant. Capsules and cartridges (made, for example, from gelatin) for use in an inhaler or insufflator can be formulated to contain a powder mix of the inhibitor and a suitable powder base such as lactose or starch. The formulations herein can be presented in unit-dose or multi-dose containers, for example sealed ampoules or vials, and can be stored in a freeze-dried (lyophilized) condition requiring only the addition of the sterile liquid carrier immediately prior to use.

Compositions in sterile pharmaceutically acceptable solvents can be nebulized by use of gases. Nebulized solutions can be breathed directly from the nebulizing device or the nebulizing device can be attached to a face mask, tent or intermittent positive pressure breathing machine. Solution, suspension, emulsion or powder compositions can be administered nasally, from devices which deliver the formulation in an appropriate amount.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir.

For topical (e.g., transdermal or transmucosal) administration, penetrants appropriate to the barrier to be permeated are generally included in the preparation. Transmucosal administration can be accomplished through the use of nasal sprays, aerosol sprays, tablets, or suppositories, and transdermal administration can be via ointments, salves, gels, patches, or creams as generally known in the art.

The term “intranasal(ly),” as used herein, refers to application of the formulations of the present invention to the surface of the skin and mucosal cells and tissues of the nasal passages, e.g., nasal mucosa, sinus cavity, nasal turbinates, or other tissues and cells which line the nasal passages. In some embodiments, intranasal administration includes administration via the nose, either with or without concomitant inhalation during administration. Such administration is typically through contact by the composition of the invention comprising the nanoemulsion with the nasal mucosa, nasal turbinates or sinus cavity. Administration by inhalation comprises intranasal administration, or can include oral inhalation. Such administration can also include contact with the oral mucosa, bronchial mucosa, and other epithelia. Such administration can also include contact with the oral mucosa, bronchial mucosa, and other epithelia. Non-limiting examples include endosinusial, endotracheal, transtracheal, intratracheal, intrabronchial, intracavernous, intrapleural, intrapulmonary, intrasinal, nasal, oral, parenteral, inhalation, subcutaneous, submucosal, mucosal, transmucosal.

Formulations according to the present disclosure can be administered in an aqueous solution as a nasal or pulmonary spray and may be dispensed in spray form by a variety of methods known to those skilled in the art. In some embodiments, the formulations herein can be presented in multi-dose containers, for example in a sealed dispensing system. Additional aerosol delivery forms can include, but are not limited to, compressed air-, jet-, ultrasonic-, and piezoelectric nebulizers, which deliver the biologically active agent dissolved or suspended in a pharmaceutical solvent, e.g., water, ethanol, or a mixture thereof.

### II. Methods

In various embodiments, the present disclosure provides methods for treating and/or preventing opioid-induced respiratory depression in a subject. The subject to be treated by the methods described herein can be a mammal. Mammals include, but are not limited to, farm animals, sport animals, pets, primates, horses, dogs, cats, mice and rats. In some embodiments, the subject to be treated by the methods described herein can be a human. In certain embodiments, a subject may have, be at risk for, or be suspected of having opioid-induced respiratory depression. Non-limiting examples of opioids that induce respiratory depression can include fentanyl, morphine, acetylfentanyl, furanylfentanyl, carfentanil, methadone, hydromorphone, alfentanil, remifentanil, sufentanil, and etorphine. A subject having or suspected of having opioid-induced respiratory depression can be identified by routine medical examination. In some examples, a subject to be treated by the methods described herein can have opioid-induced severe inspiratory flow limitation (IFL), apneas during sleep, and the like.

In some embodiments, methods disclosed herein can treat and/or prevent opioid-induced respiratory depression in a subject in need thereof who is obese. In other embodiments, methods disclosed herein can treat and/or prevent opioid-induced respiratory depression in a subject in need thereof who is overweight. In still other embodiments, methods disclosed herein can treat and/or prevent opioid-induced respiratory depression in a subject in need thereof who is of normal weight. As used herein “normal weight” can refer to a subject with a BMI of about 18.5 to about 25. As used herein “overweight” can refer to a subject with a BMI of about 25 to about 30. As used herein “obese” can refer to a subject with a BMI no less than about 30. In some aspects, the subject may be healthy. In other aspects, the subject may be recovering from injury.

In still other aspects, the subject may be recovering from surgery-induced stress. In other aspects, the subject may have a condition requiring pain management. Non-limiting examples of a condition requiring pain management include cardiovascular disease, hypertension, osteoporosis, diabetes, metabolic disorder, cancer, and the like. In other aspects, the subject may have taken at least one opioid. In some aspects, the subject may have taken a sub-lethal dose of at least one opioid. In still other aspects, the subject may be in opioid overdose. In some aspects, the subject may have leptin resistance. In some aspects, the subject may have obstructive sleep apnea.

As used herein, “an effective amount” refers to the amount of composition described herein that confers a therapeutic effect on the subject, either alone or in combination with one or more other active agents. Determination of whether an amount of the composition disclosed herein achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. Generally, a maximum dose of the individual components or combinations thereof is that which can be used, that is, to the highest safe dose according to sound medical judgment.

In some embodiments, an “effective amount” of a composition herein is the amount of the composition that alone, or together with further doses, produces the desired response, e.g., attenuates one or more effects of opioid-induced respiratory depression. In some embodiments, methods of administering a composition that includes leptin can attenuate one or more effects of opioid-induced respiratory depression. In some aspects, one or more effects of opioid-induced respiratory depression can include increased IFL breath frequency, increased apneas, decreased maximal inspiratory flow (V_{Imax}), decreased minute ventilation (V_{E}), decreased tidal volume, increased dead space, decreased upper airway patency, or a combination thereof.

In some examples, methods disclosed herein can increase the breathing rate of the subject in need thereof after administering leptin compared to an untreated subject with identical disease condition and predicted outcome. In some examples, methods disclosed herein can increase the breathing rate in a subject in need thereof by at least about 10%. In some examples, methods disclosed herein can increase the breathing rate in a subject in need thereof by about 10% to about 100% (e.g., about 10%, about 20%, about 30%, about 40%, about 50%, about 60% about 70%, about 80%, about 90%, or about 100%).

In some examples, methods disclosed herein can decrease apneas of the subject in need thereof after administering leptin compared to an untreated subject with identical disease condition and predicted outcome. In some examples, methods disclosed herein can decrease apneas in a subject in need thereof by at least about 10%. In some examples, methods disclosed herein can decrease apneas in a subject in need thereof by about 10% to about 100% (e.g., about 10%, about 20%, about 30%, about 40%, about 50%, about 60% about 70%, about 80%, about 90%, or about 100%). In some examples, methods disclosed herein can improve obstructive sleep apnea of a subject in need thereof after administering leptin compared to an untreated subject with identical disease condition and predicted outcome. In some examples, methods disclosed herein can improve obstructive sleep apnea in a subject in need thereof by at least about 10%. In some examples, methods disclosed herein can improve obstructive sleep apnea in a subject in need thereof by about 10% to about 100% (e.g., about 10%, about 20%, about 30%, about 40%, about 50%, about 60% about 70%, about 80%, about 90%, or about 100%).

In some examples, methods disclosed herein can increase maximum inspiratory flow rate (V_{Imax}) of the subject in need thereof after administering leptin compared to an untreated subject with identical disease condition and predicted outcome. In some examples, methods disclosed herein can increase maximal inspiratory flow in a subject in need thereof by at least about 10%. In some examples, methods disclosed herein can increase maximal inspiratory flow in a subject in need thereof by about 10% to about 100% (e.g., about 10%, about 20%, about 30%, about 40%, about 50%, about 60% about 70%, about 80%, about 90%, or about 100%).

In some examples, methods disclosed herein can increase minute ventilation of the subject in need thereof after administering leptin compared to an untreated subject with identical disease condition and predicted outcome. In some examples, methods disclosed herein can increase minute ventilation in a subject in need thereof by at least about 10%. In some examples, methods disclosed herein can increase minute ventilation in a subject in need thereof by about 10% to about 100% (e.g., about 10%, about 20%, about 30%, about 40%, about 50%, about 60% about 70%, about 80%, about 90%, or about 100%).

In some examples, methods disclosed herein can increase tidal volume of the subject in need thereof after administering leptin compared to an untreated subject with identical disease condition and predicted outcome. In some examples, methods disclosed herein can increase tidal volume in a subject in need thereof by at least about 10%. In some examples, methods disclosed herein can increase tidal volume in a subject in need thereof by about 10% to about 100% (e.g., about 10%, about 20%, about 30%, about 40%, about 50%, about 60% about 70%, about 80%, about 90%, or about 100%).

In some examples, methods disclosed herein can decrease dead space of the subject in need thereof after administering leptin compared to an untreated subject with identical disease condition and predicted outcome. In some examples, methods disclosed herein can decrease dead space in a subject in need thereof by at least about 10%. In some examples, methods disclosed herein can decrease dead space in a subject in need thereof by about 10% to about 100% (e.g., about 10%, about 20%, about 30%, about 40%, about 50%, about 60% about 70%, about 80%, about 90%, or about 100%).

In some examples, methods disclosed herein can increase upper airway patency of the subject in need thereof after administering leptin compared to an untreated subject with identical disease condition and predicted outcome. In some examples, methods disclosed herein can increase upper airway patency in a subject in need thereof by at least about 10%. In some examples, methods disclosed herein can increase upper airway patency in a subject in need thereof by about 10% to about 100% (e.g., about 10%, about 20%, about 30%, about 40%, about 50%, about 60% about 70%, about 80%, about 90%, or about 100%).

In some embodiments, an “effective amount” of a composition herein is the amount of the composition that alone, or together with further doses, produces the desired response, e.g., reverses the effect of opioids in the hypoglossal motor neurons (HGNs). In some embodiments, methods of administering a composition that includes leptin can reverse the effect of opioids in the hypoglossal motor neurons (HGNs). In some aspects, methods of administering leptin restores the HGN firing rate that was dampened by opioid administration. In some examples, methods disclosed herein can restore the HGN firing rate that was dampened by opioid administration in a subject in need thereof by at least about 10%. In some examples, methods disclosed herein can restores the HGN firing rate that was dampened by opioid administration in a subject in need thereof by about 10% to about 100% (e.g., about 10%, about 20%, about 30%, about 40%, about 50%, about 60% about 70%, about 80%, about 90%, or about 100%). In other aspects, methods of administering leptin restores the excitatory post-synaptic current frequency in HGNs that was dampened by opioid administration. In some examples, methods disclosed herein can restore the excitatory post-synaptic current frequency in HGNs that was dampened by opioid administration in a subject in need thereof by at least about 10%. In some examples, methods disclosed can restore the excitatory post-synaptic current frequency in HGNs that was dampened by opioid administration in a subject in need thereof by about 10% to about 100% (e.g., about 10%, about 20%, about 30%, about 40%, about 50%, about 60% about 70%, about 80%, about 90%, or about 100%).

Empirical considerations, such as the half-life, generally will contribute to the determination of the dosage. Frequency of administration can be determined and adjusted over the course of therapy, and is generally, but not necessarily, based on treatment and/or suppression and/or amelioration and/or delay of a target disease/disorder. Alternatively, sustained continuous release nasal formulations can be appropriate. Various formulations and devices for achieving sustained release are known in the art.

In some other embodiments, leptin can be administered intranasally once about every 1 hours to about 24 hours. In some aspects, leptin may be administered intranasally once. In some other aspects, leptin can be administered intranasally in combination with at least one opioid. In other aspects, leptin can be administered intranasally in combination with at least one opioid for up to 5 days following surgery. In still other aspects, leptin can be administered intranasally immediately following an overdose of at least one opioid. As used herein, “immediately following an overdose” can be within about 1 minute to about 15 minutes of a subject exhibiting at least one symptom of an opioid overdose. In some aspects, a symptom of an opioid overdose can be loss of consciousness, unresponsive to outside stimulus, inability to speak, slow and shallow, erratic, or no breathing, change in skin tone, emitting a choking sound, vomiting, limp body posture, very pale or clammy face, pulse is slow, erratic, or absent, or a combination thereof

In some embodiments, leptin can be administered intranasally to a subject in need thereof using any of the compositions described herein. In some embodiments, methods of administering leptin can include administering a nasal formulation that includes at least leptin as described herein. Dosages of nasal formulations as described herein can be determined empirically in individuals who have been given one or more administration(s) of the formulation. Generally, for administration of any of the nasal formulations described herein, an initial candidate dosage can be about 0.01 mg/ml leptin to about 10.0 mg/ml or more leptin depending on the factors mentioned above. For repeated administrations over several days or longer, depending on the condition, the treatment can be sustained until a desired suppression of symptoms occurs or until sufficient therapeutic levels are achieved to alleviate a target disease or disorder, or a symptom thereof. In some embodiments, dosage regimens can be useful, depending on the pattern of pharmacokinetic decay that the practitioner wishes to achieve. For example, dosing from about one to seven times a week is contemplated. In some embodiments, dosing ranging from about 0.01 mg/ml to about 20.0 mg/ml leptin per day can be used. In some embodiments, dosing frequency is three times a day or more, two times a day, once every day, every other day, twice a week, once week, every 2 weeks, every 4 weeks, every 5 weeks, every 6 weeks, every 7 weeks, every 8 weeks, every 9 weeks, or every 10 weeks; or once every month, every 2 months, or every 3 months, or longer or whenever one or more effects of opioid-induced respiratory depression is attenuated.

The progress of this therapy is easily monitored by conventional techniques and assays. The dosing regimen can vary over time.

In some embodiments, an appropriate dosage of a nasal formulation as described herein can depend on the type and severity of the disease/disorder, whether the formulation is administered for preventive or therapeutic purposes, previous therapy, the subject's clinical history and response to the formulation, and the discretion of the attending physician. Typically the clinician will administer the disclosed nasal formulation, until a dosage is reached that achieves the desired result. In some embodiments, the desired result is decrease in the intracranial pressure in the subject. In some examples, the desired result is an about 1% to an about 100% decrease in opioid-induced respiratory depression in a subject after administration of a nasal formulation disclosed herein. In some examples, the desired result is an about 1% to an about 100% decrease in at least one symptom of an opioid overdose in a subject after administration of a nasal formulation disclosed herein. Methods of determining whether a dosage resulted in the desired result would be evident to one of skill in the art. Administration of the nasal formulations herein can be continuous or intermittent, depending, for example, upon the recipient's physiological condition, whether the purpose of the administration is therapeutic or prophylactic, and other factors known to skilled practitioners. The administration of the nasal formulations herein can be essentially continuous over a preselected period of time or may be in a series

In some embodiments, methods of treating a subject can include the step of administering the formulations disclosed herein intranasally to a subject in need thereof. In some examples, a formulation can be administered to a subject via nasal spray, a metering, atomizing spray pump. Each actuation of the pump delivers a single dosage of the drug substance to the subj ect.

In various embodiments, the leptin and compositions and formulations containing leptin as disclosed herein may be utilized in conjunction with other types of therapy for opioid-induced respiratory depression, opioid overdose, or a combination thereof. In some embodiments, the leptin and compositions and formulations containing leptin as disclosed herein may be utilized in conjunction with naloxone, naloxone related substances, any of their pharmaceutically acceptable salts, or a combination thereof In some aspects, methods herein can be used for treating a subject in need thereof wherein the subject was treated with naloxone, naloxone related substances, any of their pharmaceutically acceptable salts, or a combination thereof In some examples, compositions described herein can be administered before, during, or after administration of at least one naloxone, naloxone related substances, any of their pharmaceutically acceptable salts, or a combination thereof. Additional useful agents and therapies for opioid-induced respiratory depression, opioid overdose, or a combination thereof can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

In various embodiments, methods of administering a composition that includes leptin and naloxone can act in a synergistic manner to attenuate one or more effects of opioid-induced respiratory depression. In some examples, methods of administering a composition that includes leptin and naloxone can act in a synergistic manner to attenuate increased IFL breath frequency, increased apneas, decreased maximal inspiratory flow (V_{Imax}), decreased minute ventilation (V_{E}), decreased tidal volume, increased dead space, decreased upper airway patency, or a combination thereof.

In various embodiments, methods of administering a composition that includes leptin and naloxone can act in an additive manner to attenuate one or more effects of opioid-induced respiratory depression. In some examples, methods of administering a composition that includes leptin and naloxone can act in an additive manner to attenuate increased IFL breath frequency, increased apneas, decreased maximal inspiratory flow (V_{Imax}), decreased minute ventilation (V_{E}), decreased tidal volume, increased dead space, decreased upper airway patency, or a combination thereof.

In various embodiments, methods of administering a composition that includes leptin as disclosed herein can replace the need for administering naloxone in the subject in need thereof. Non-limiting examples of a need for administering naloxone can include treating an acute opioid overdose, treating respiratory or mental depression due to opioid use,

### III. Kits

Compositions of the present invention may, if desired, be presented in a pack or dispenser device, such as an FDA approved kit, which may contain one or more unit dosage forms containing the active ingredient. The pack can, for example, comprise metal or plastic foil, such as a blister pack. The pack or dispenser device can be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In some embodiments, kits containing nasal formulations disclosed herein and at least one container are contemplated. In other embodiments, kits can include instructions for use in accordance with any of the methods described herein. The included instructions can comprise a description of administration of the pharmaceutical composition for delivering the therapeutic agent or diagnostic agent encapsulated therein or for treating opioid-induced respiratory depression, opioid overdose, or the like according to any of the methods described herein. The kit may further include a description of selecting an individual suitable for treatment based on identifying whether that individual has, is suspected of having, or is at risk for opioid-induced respiratory depression, opioid overdose, or the like.

In some embodiments, instructions relating to the use of the nasal formulations described herein, generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. In some embodiments, kits as described herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or any suitable device for nasal delivery.

In some embodiments, compositions disclosed herein can be included in an emergency overdose response kit.

### EXAMPLES

The following examples are included to demonstrate preferred embodiments of the disclosure. It should be appreciated by those of skill in the art that the techniques disclosed in the examples that follow represent techniques discovered by the inventors to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1

### Subcutaneous Leptin Stimulates Breathing in Lean Mice.

Leptin increases minute ventilation, stimulates control of breathing and reverses UAO during sleep in leptin deficient ob/ob mice as shown in in Pho et al., Journal of Applied Physiology 120, 78-86 (2016); Yao et al., Sleep 39, 1097-106 (2016); and O'Donnell et al., American Journal of Respiratory & Critical Care Medicine 159, 1477-1484 (1999), the disclosures of which are incorporated herein in their entirety. As such, it was proposed that high doses of leptin also stimulate ventilation and control of breathing in lean C57BL/6J mice. To assess this, male C57BL/6J mice, 25.4±0.7 g of weight (n=12) were first acclimated for 4-7 days to the barometric plethysmography chamber as described in Caballero-Eraso et al., The Journal of Physiology 597, 151-172 (2019), the disclosure of which is incorporated herein in its entirety. In six mice, a subcutaneous osmotic pump (DURECT, Cupertino, Calif., USA) filled with saline was implanted and baseline minute ventilation (V_{E}) and the hypoxic ventilatory response (HVR) were measured by exposing mice to room air and 10% O₂. The measurements were repeated 2-3 times and then the pump was removed. V_{E} normalized to body weight and the hypoxic ventilatory response (HVR) was calculated as the ratio of [V_{E} (10% O₂)−V_{E} (20.9% O₂) to the change in FiO₂ (ΔFiO₂=10.9% between normoxia and hypoxia) and reported as ΔV_{E}/ΔFiO₂. Subsequently, a subcutaneous osmotic pump for leptin infusion (0.2 mg/kg/hour) was implanted and the HVR was measured. In six other mice the order (saline vs. leptin pump) was reversed. All measurements were performed during wakefulness and, therefore, inspiratory flow limitation indicating upper airway obstruction (UAO) was absent. Infusion of leptin increased V_{E}, from 1.1 to 1.5 ml/minutes/grams, and the hypoxic ventilatory response, from 0.23 to 0.31 ml/minutes/grams/ΔFiO₂.

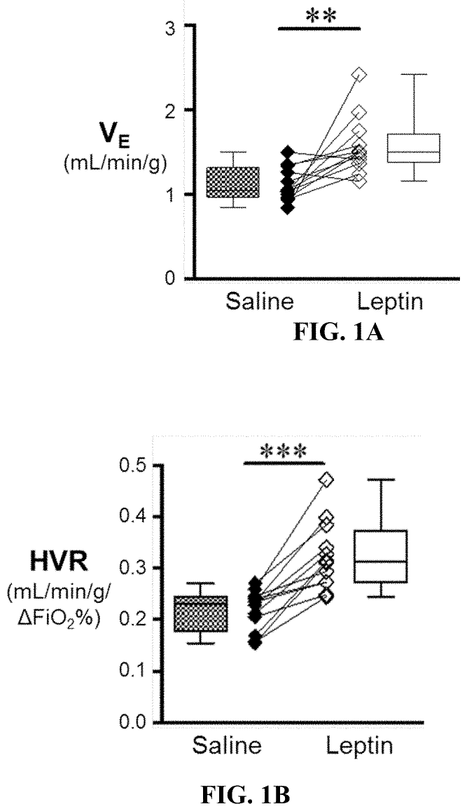

As shown in FIG. 1A, subcutaneous leptin increased minute ventilation (V_{E}) in lean C57BL/6J mice. Also, FIG. 1B shows subcutaneous leptin increased the hypoxic ventilatory response (HVR) in lean C57BL/6J mice. The data demonstrate that leptin acted as a powerful respiratory stimulant in lean mice. Further, data support that synthetic opiates induce opiate-mediated respiratory suppression (ORS) by both suppressing control of breathing and upper airway patency and that intranasal leptin can treat both types of ORS in lean mice.

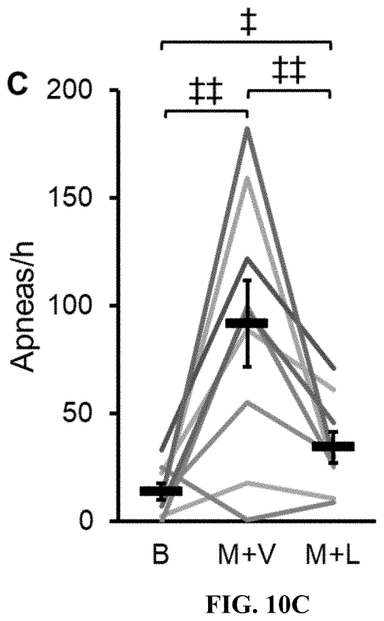

To determine the effect of intranasal (IN) leptin in the brain, leptin detection in brain tissue by ELISA was assessed after IN administration. In brief, male lean C57BL/6J mice (30 g of weight), were treated with IN leptin (24 μl) at 0.8 mg/kg in 1% BSA or 1% BSA and sacrificed 20 minutes later. Brains were extracted, then the olfactory bulbs, hypothalami and medullas were isolated, quick frozen in liquid nitrogen and stored at −80° C. For leptin level measurements, brain tissue was homogenized in 160 mM KCL, 25 mM HEPES, 0.2% Triton X-100 and protease inhibitors. Total protein concentrations were determined using a BioRad DC Protein kit and ELISA for leptin was performed with a Millipore kit. As shown in FIGS. 8A-8C, IN leptin administered at the dose effective for treating ORS significantly increased leptin levels in the olfactory bulb and medulla with a strong increase in the hypothalamus. These data showed that IN leptin increased leptin levels in the brain, which was detectable by ELISA, after IN administration of leptin.

### EXAMPLE 2

### Intranasal Leptin Treats Sleep Disordered Breathing in Diet-Induced Obese Mice

Diet-induced obese (DIO) mice are leptin resistant and develop awake hypercapnea and sleep disordered breathing, for example obstructive sleep apnea (OSA). To assess effects of intranasal (IN) delivery of leptin on leptin resistance a mouse model of OSA was used, wherein the mouse model is described in Pho et al., Journal of Applied Physiology 120, 78-86 (2016); Yao et al., Sleep 39, 1097-106 (2016); Fleury Curado et al., Sleep zsy089-zsy089 (2018); and Berger et al., Am J Respir Crit Care Med (2018), the disclosures of which are incorporated herein in their entirety. The mouse OSA model is ideal because despite differences in the upper airway anatomy between mice and humans, they share many essential similarities. For example, the most collapsible upper airway segment in mice is the velopharynx, like in humans, and upper airway function and treatment responses to stimulation of the hypoglossal motoneurons (HGN) and lingual muscles are comparable between two species. Used herein are plethysmographic methods for monitoring high-fidelity airflow and respiratory effort signals continuously during sleep study in mice. Upper airway obstruction was defined by the presence of inspiratory airflow limitation (IFL) characterized by an early inspiratory plateau in airflow at a maximum level (Vᵢₘₐₓ) while effort continued to increase. Because IFL is a cardinal feature of upper airway obstruction during sleep in humans who snore and have obstructive sleep apnea-hypopnea syndrome, measuring IFL in the mouse model is a valid assessment for clinical applicability in humans.

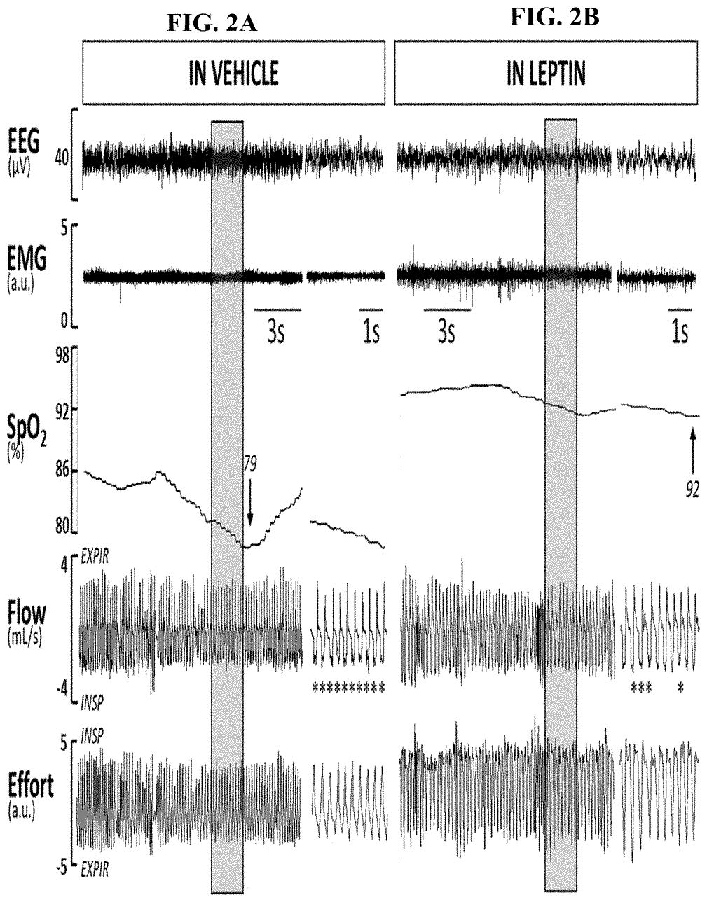

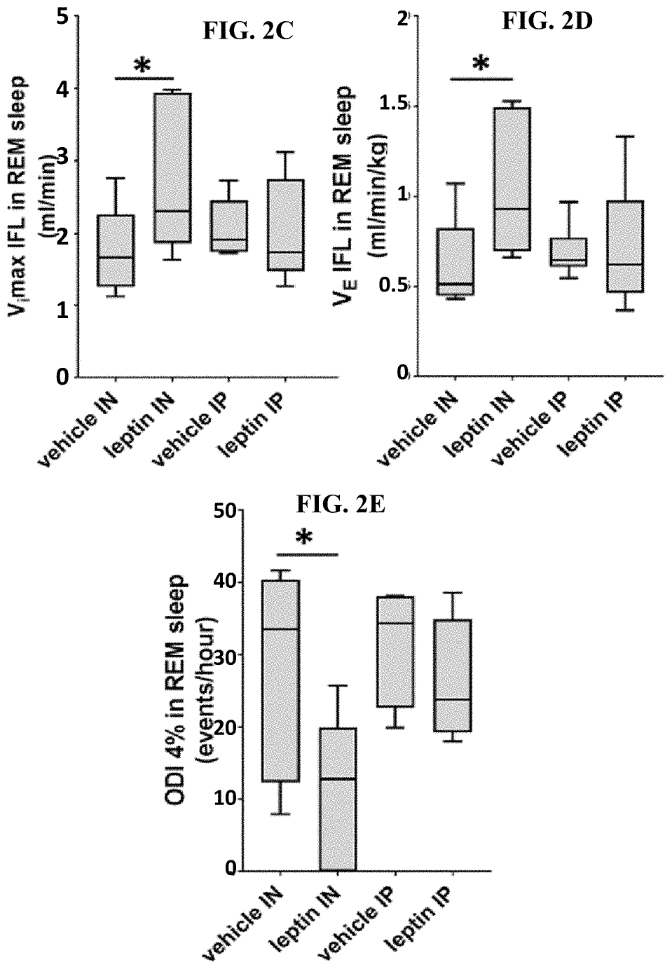

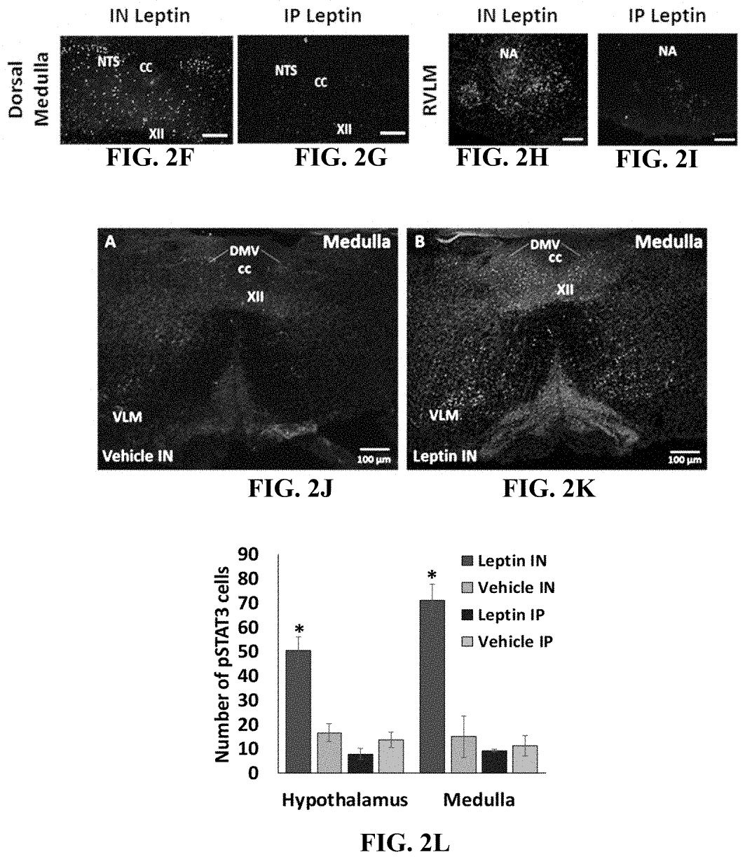

To determine if intranasal (IN) delivery of leptin will circumvent leptin resistance, male DIO C57BL/6J mice, 43.3 ±5.8 g of weight that were housed at 12 hour light cycle (lights on at 9 AM) were treated with a single dose of intranasal (IN) or intraperitoneal (IP) leptin (0.4 mg/kg in 24 μl of bovine serum albumin (BSA)) vs BSA, n=10, in a cross-over manner 1 week apart at 10:30 AM. Sleep studies were performed in previously acclimated animals from 11 AM to 5 PM. Shown in FIGS. 2A and 2B are representative REM sleep recordings in a DIO mice treated with IN vehicle (FIG. 2A) or IN leptin (FIG. 2B). Non-flow minute ventilation (NFL) and flow limited breathing (IFL) reflecting upper airway obstruction during sleep (OSA) were scored separately. After sleep studies, mice recovered for a week and then were treated with a single dose of IN/IP leptin or vehicle and sacrificed one hour later to measure leptin signaling by STAT3 phosphorylation (pSTAT3) in the dorsal & rostral ventral lateral medulla (RVLM) by performing immunofluorescent staining using a general method as described in, for example, Ausubel, SHORT PROTOCOLS IN MOLECULAR BIOLOGY: A COMPENDIUM OF METHODS FROM CURRENT PROTOCOLS IN MOLECULAR BIOLOGY. West Sussex: J. Wiley, 2002, the disclosure of which is incorporated herein in its entirety.

IN leptin increased Vᵢₘₐₓ (FIG. 2C) and V_{E} (FIG. 2D) during flow limited obstructed breathing treating OSA, which resulted in a dramatic decrease in ODI (a number of oxyhemoglobin desaturations ≥4%/hour)(FIG. 2E). IN leptin also increased non-flow limited breathing and signaling (STAT3 phosphorylation) in the dorsal medulla (FIG. 2F), rostral ventral lateral medulla (RVLM) (FIG. 2H), central canal (CC), dorsal motor nucleus of the vagus (DMV), nucleus ambiguous (NA) and hypoglossal nucleus (XII) (FIG. 2K) via the long isoform of leptin receptor, LepR^{b}. In contrast, IP leptin had no effect in either the dorsal medulla (FIG. 2G) or RVLM (FIG. 2I). Further, vehicle administered intranasally (IN) did not increase non-flow limited breathing and signaling (STAT3 phosphorylation) in the dorsal & rostral ventral lateral medulla (VLM), central canal (CC), dorsal motor nucleus of the vagus (DMV), nucleus ambiguous (NA) or hypoglossal nucleus (XII) (FIG. 2J). FIG. 2L shows the number of pSTAT3-positive cells in the hypothalamus and medulla were highest in IN leptin-treated mice compared to the other experimental groups.

Collectively, these data show that in obese mice, IN leptin treats OSA and hypoventilation by circumventing BBB and acting on medullary centers controlling upper airway patency.

### EXAMPLE 3

### Morphine Induces Obstructive Sleep Apnea in Diet-Induced Obese Mice, which can be Prevented by Intranasal Leptin

To evaluate whether leptin prevents opiate-mediated respiratory suppression (ORS), the effect of leptin on opiate-induced suppression of ventilatory control was assessed by measuring non-flow limited breathing and on upper airway obstruction (UAO) during morphine-induced sedation induced by either a 10 mg/kg dose of morphine or an 80 mg/kg dose of morphine.

In brief, male DIO C57BL/6J mice (45.0±2.0 grams, n=4) had a baseline sleep study and then were treated with IN leptin or IN BSA in a cross-over fashion one week apart as described in Example 2 above. Thirty minutes after IN treatment, the morphine bolus was injected intraperitoneally (IP) at 10 mg/kg. Given that a half-life of morphine in DIO mice is about 90 minutes, morphine was subsequently infused subcutaneously (SC) via osmotic pump at 2 mg/kg/hour and sleep studies were performed as described in the previous examples herein. Expression of μ opiate receptor (MOR) and LepR^{b} on hypoglossal motoneurons (HGN) innervating genioglossus (GG) muscle was examined after injection of retrograde tracer cholera toxin B (CTB) to the GG muscle.

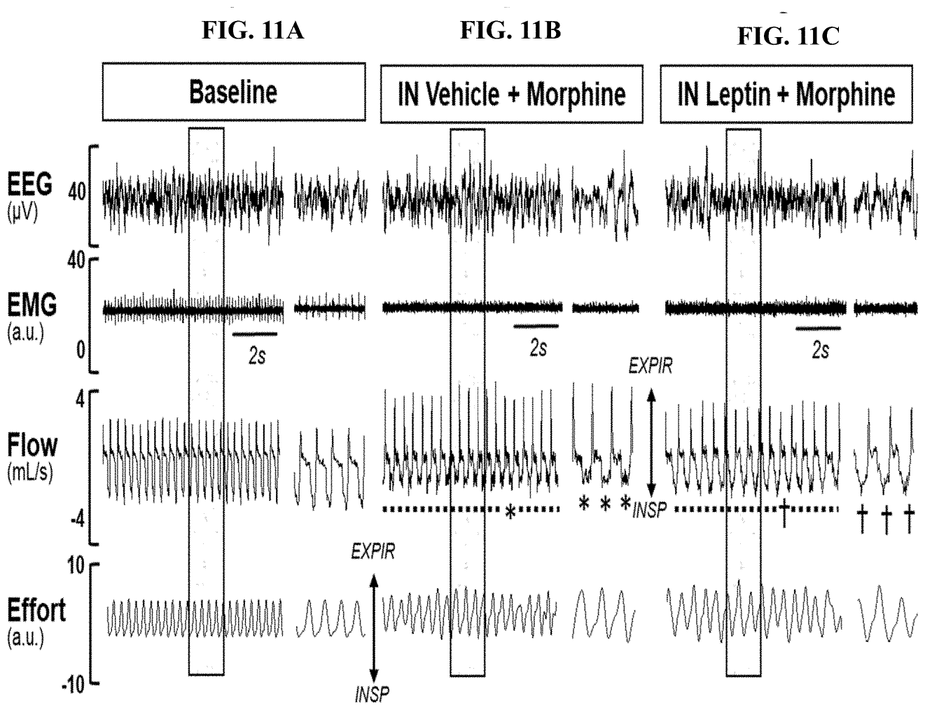

During wakefulness morphine reduced minute ventilation (from 2.3±0.6 ml/min/g to 1.6±0.7 ml/min/g; F_{2,6}=4.145; partial eta²=0.34; p<0.05). Continuous movement induced by morphine interfered with the respiratory analysis when mice were awake. IN leptin was administered 30 minutes prior to morphine; animals were awake and moving after leptin instillation, therefore the precise onset of leptin's respiratory effects could not be established. Similarly, wakefulness in morphine-treated mice was characterized by constant activity, which masked effects of leptin. In awake morphine-treated mice minute ventilation was 1.6±0.6 ml/min/g in the absence and 1.8 ±0.6 ml/min/g in the presence of leptin (p>0.05). The analysis of breathing during sleep showed that morphine induced upper airway obstruction and suppressed both non-flow limited and flow limited breathing throughout the entire 6 hour recording (FIGS. 9A-9E).

Morphine increased NREM sleep and eliminated REM sleep (Table 1). Leptin significantly increased sleep efficiency and consolidated NREM sleep in morphine-treated mice, increasing the duration of NREM bouts and decreasing the number of NREM bouts (Table 1).

TABLE 1
Characteristics of mice, sleep architecture, and apnea classification

| | | | Morphine + IN | Morphine + IN |
|---|---|---|---|---|
| | | Baseline | Vehicle | Leptin |
| Body weight | (g) | 43.1 ± 1.4 | 44.0 ± 1.4 | 44.1 ± 1.6 |
| Age | (weeks) | 24.1 ± 1.4 | 24.3 ± 1.6 | 24.5 ± 1.4 |
| Sleep Efficiency | (% of total time) | 63.7 ± 3.5 | 84.3 ± 3.2 | 92.3 ± 1.0 |
| REM sleep | % of total sleep time | 2.3 ± 0.1 | 0 | 0 |
| | Number of bouts | 3.1 ± 0.7 | 0 | 0 |
| | Duration of bout (min) | 1.2 ± 0.2 | 0 | 0 |
| NREM | % of total sleep time | 97.7 ± 0.7 | 100 | 100 |
| | Number of bouts | 47.3 ± 9.7 | 37 ± 8.3 | 18 ± 3.0 |
| | Duration of bout (min) | 4.3 ± 0.6 | 9.63 ± 1.9 | 20.5 ± 4.1 |
| Apnea index by type | Obstructive (/h) | 1.3 ± 0.6 | 9.2 ± 3.9 | 6.1 ± 1.6 |
| | Central (/h) | 7.9 ± 2.0 | 12.8 ± 3.4 | 11.8 ± 1.9 |
| | Unidentified (/h) | 3.0 ± 1.4 | 41.9 ± 8.6 | 24.1 ± 7.8 |

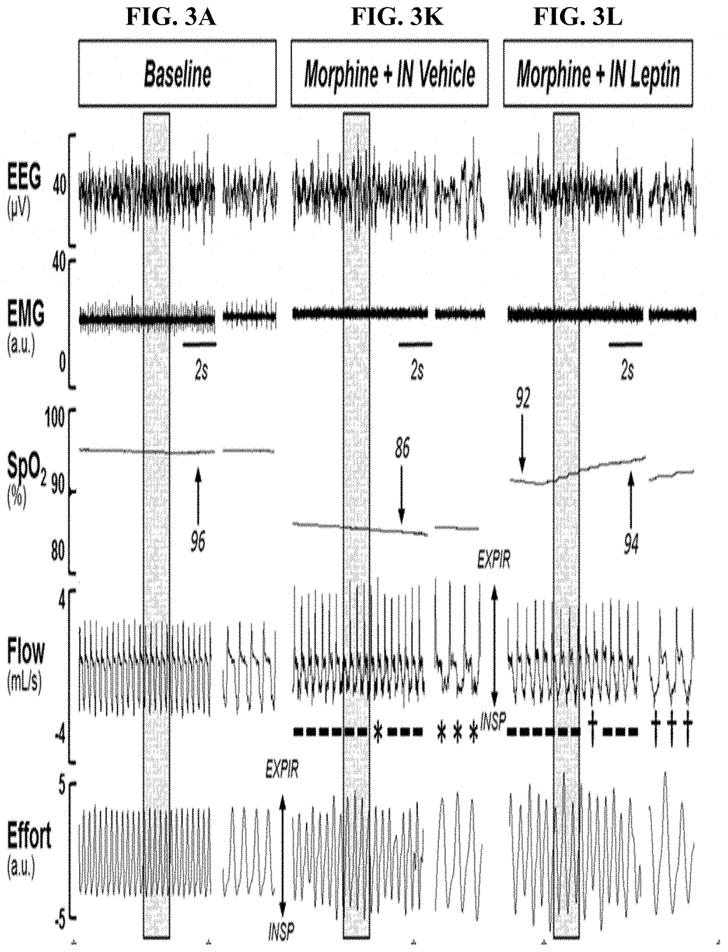

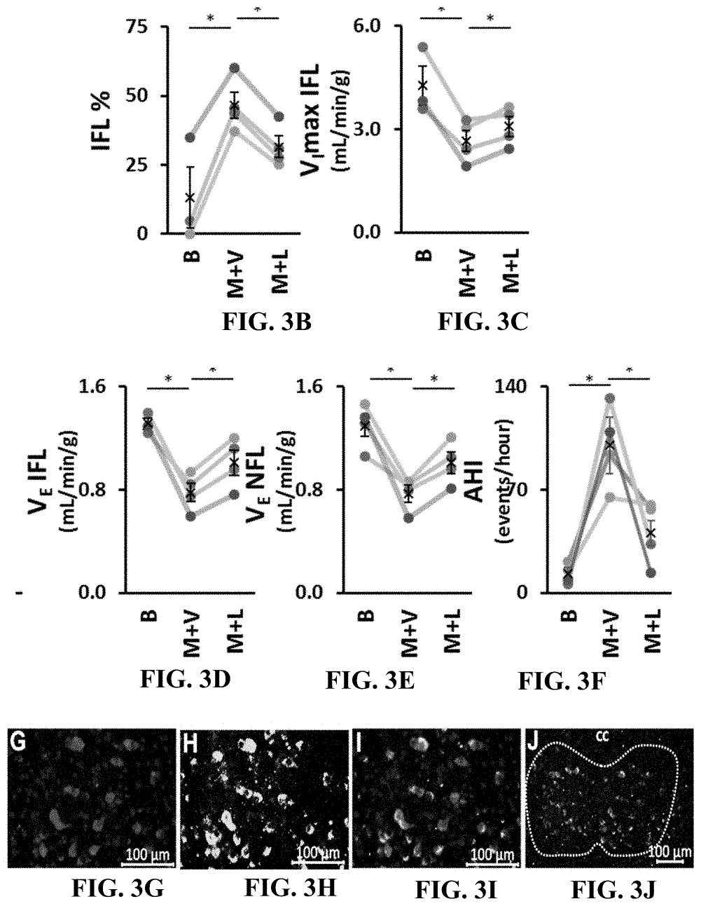

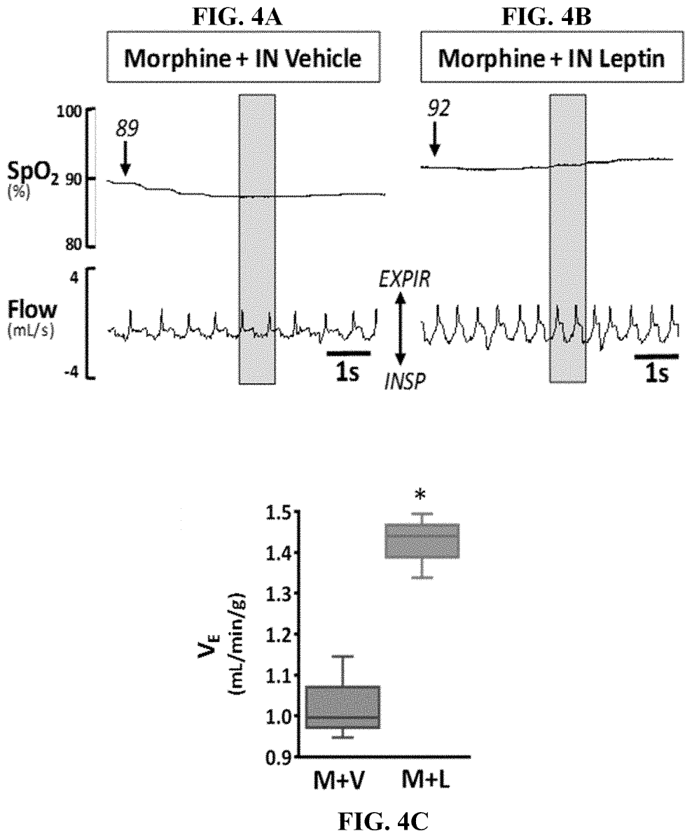

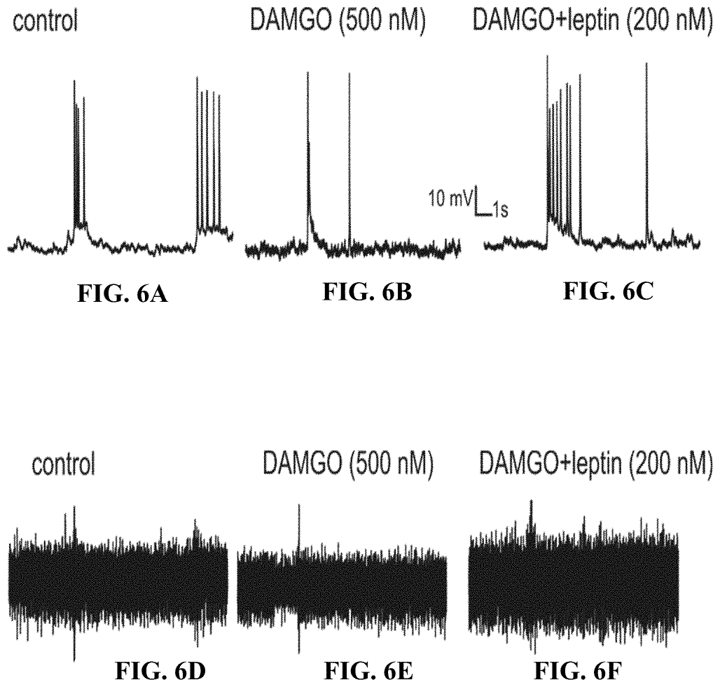

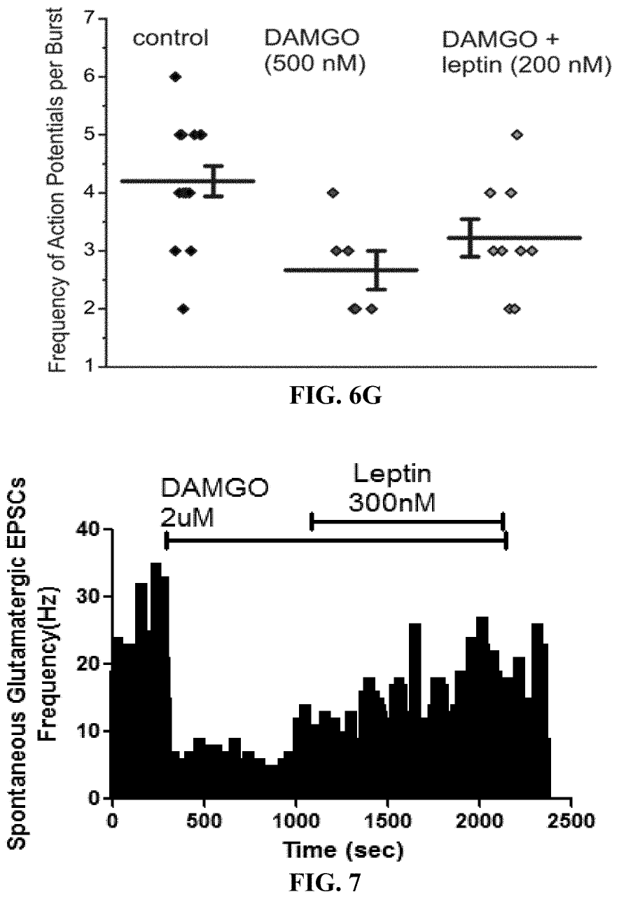

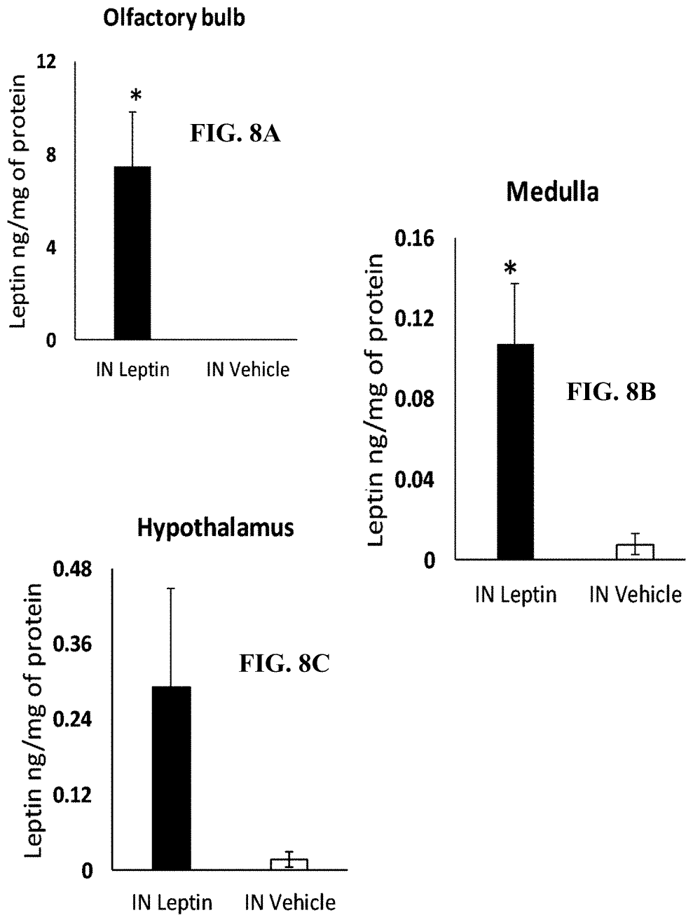

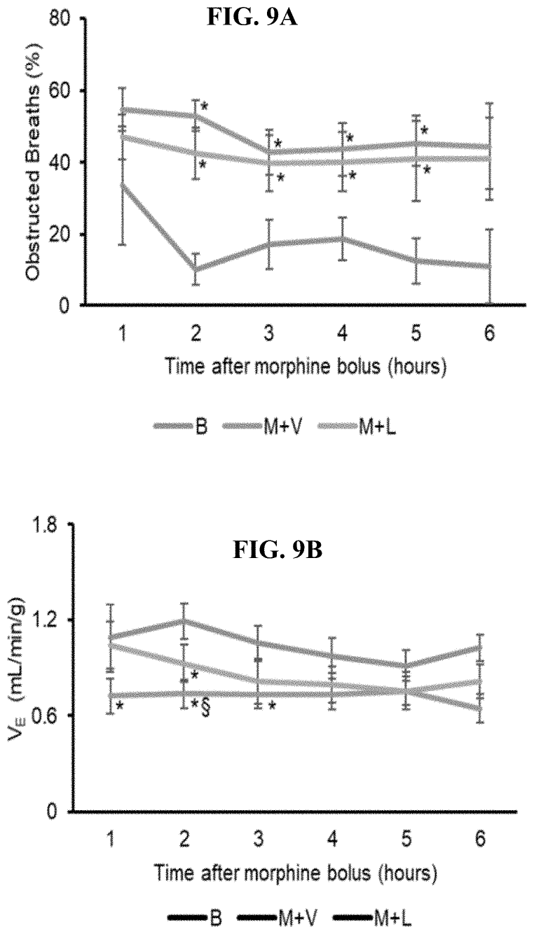

Shown in FIGS. 3A, 3K and 3L are representative REM sleep recordings showing a typical recording at baseline (FIG. 3A), a typical recording after administering IN vehicle+morphine at 10 mg/kg (FIG. 3K), and a typical recording after administering IN leptin+morphine (FIG. 3L). Collectively, FIGS. 3A, 3K and 3L demonstrate that opiates induced severe inspiratory flow limitation (IFL) and upper airway obstruction (OSA) leading to hypoxemia during morphine-induced sedation. Morphine increased IFL prevalence to 39.6±9.3% of all breaths (FIG. 3B) decreasing Vᵢₘₐₓ and V_{E} (FIGS. 3C and 3D). Also, morphine also suppressed non-flow limited breathing (NFL) which decreased respiratory rate (FIG. 3E). There was a significant increase in the apnea-hypopnea index (AHI)(FIG. 3F) due to both central events (predominantly apneas) and obstructive (predominantly hypopneas). IN leptin decreased frequency (FIG. 3B) and relieved severity of upper airway obstruction induced by morphine, which was manifested by increases in maximal inspiratory flow (Vᵢₘₐₓ) and minute ventilation during IFL (V_{E} IFL) (FIGS. 3C and 3D). IN leptin prevented respiratory depression during NFL breathing (FIG. 3E) increasing tidal volume (from 0.36±0.04 to 0.44±0.03, p<0.05), whereas respiratory rate did not change. Leptin dramatically reduced AHI (FIG. 3F). MORs were abundant in the hypoglossal motoneurons (HGN) innervating GG (FIGS. 3G-3J). Although LepR^{b} is not expressed on HGN, abundant projections from LepR^{b}+ neurons to HGNs were detected (FIG. 5B).

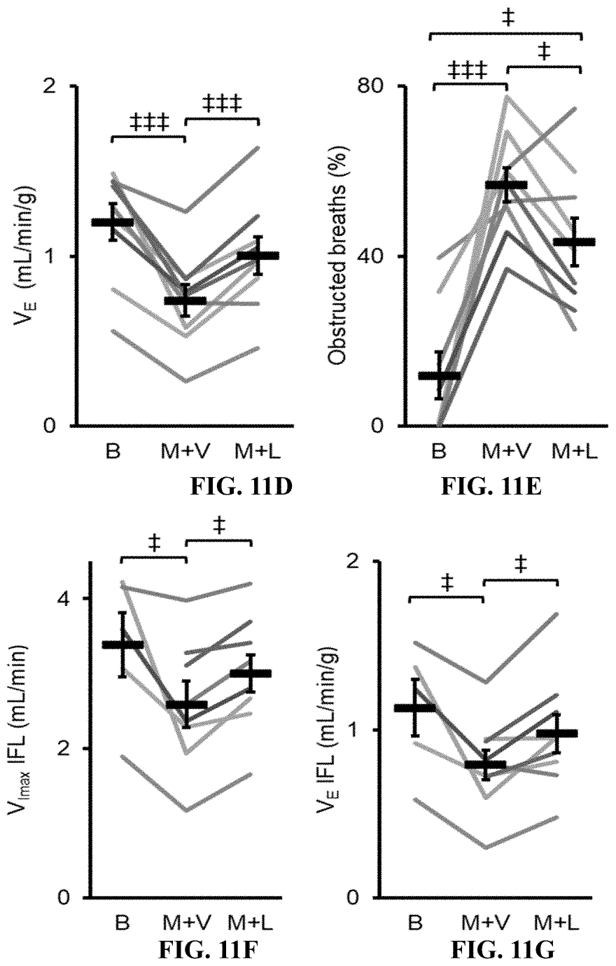

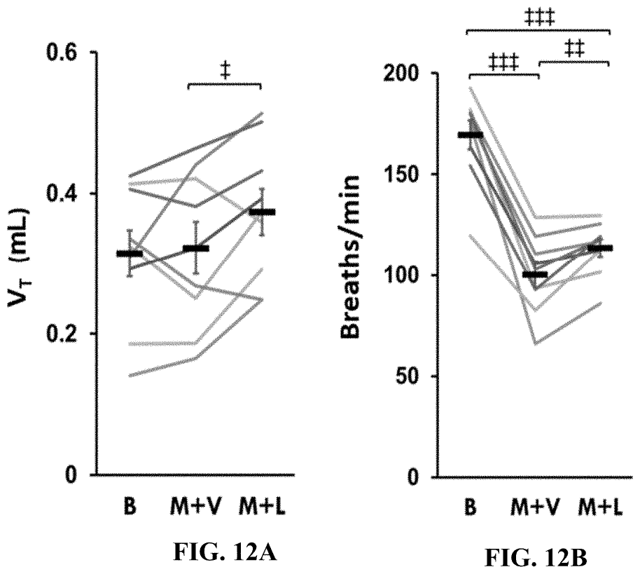

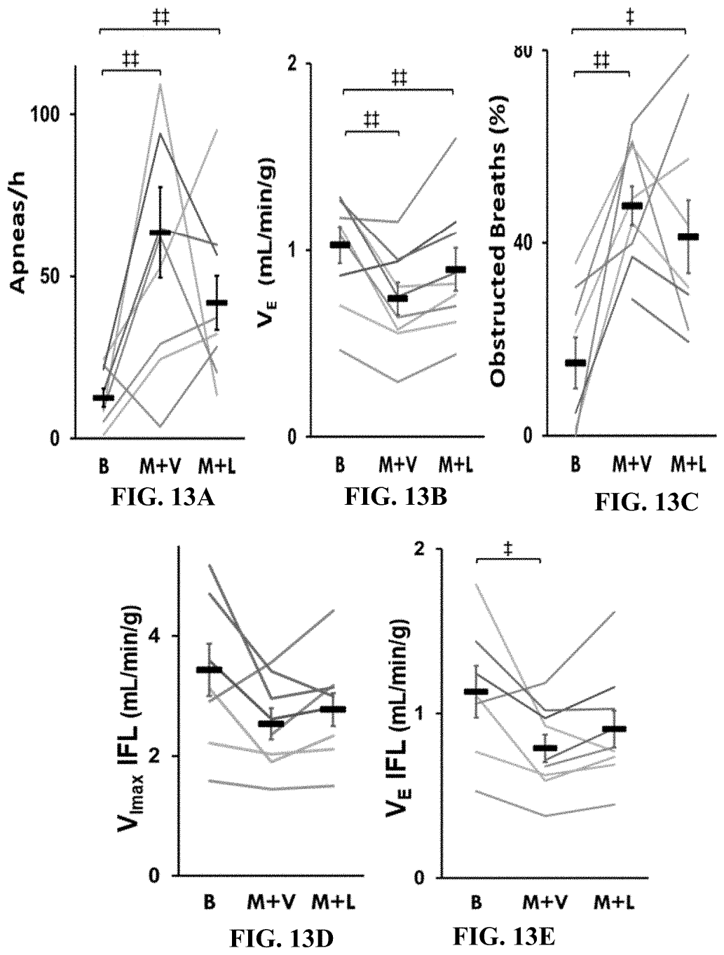

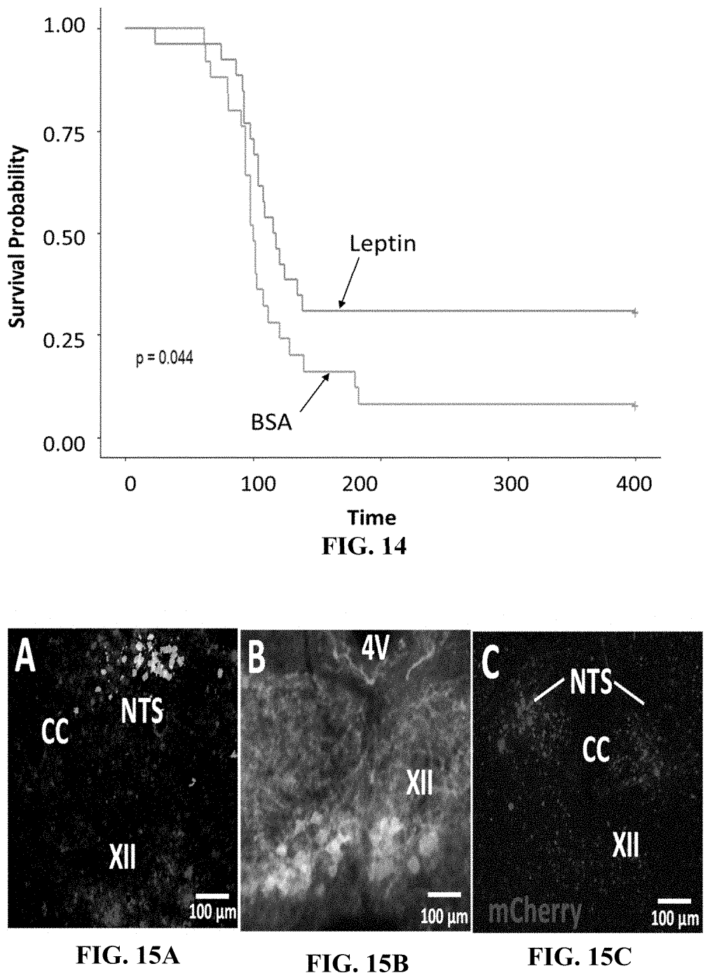

Morphine dramatically increased the number of apneic events during NREM sleep, from 13.9±3.7 to 91.5±20.0 (F_{2,6}=12.365; partial eta²=0.61; p=0.006) (FIGS. 10A-10C). Apneas were classified as obstructive, characterized by cessation of airflow in the presence of respiratory effort, or central, in which the effort was absent. In some instances, effort could not be quantified, and therefore these events were labeled as unidentified (Table 1). The effect of morphine on control of breathing was evident during breathing in the absence of upper airway obstruction, i.e. non-flow limited respiration. As expected, morphine suppressed minute ventilation (from 1.2±0.1 ml/min/g to 0.7±0.1 ml/min/g; F_{2,6}=20.593; partial eta²=0.72; p<0.001) decreasing respiratory (FIGS. 11A-11G, FIG. 12A, and FIG. 12B). The breath-by-breath analysis also showed that morphine dramatically increased frequency of upper airway obstruction, defined by inspiratory flow limitation (IFL) with a plateau in early inspiration, from 11.9±5.5% to 56.8±4.8% of all breaths (F_{2,5}=17.149; partial eta²=0.71; p<0.001) (FIGS. 11A, 11B, and 11E). In fact, three of nine mice did not have any obstructed breaths during NREM sleep at baseline, whereas all mice demonstrated markedly increased upper airway obstruction during morphine treatment. The comparative analysis of obstructed breaths in five mice exhibiting inspiratory flow limitation at baseline and during morphine treatment indicated that the severity of upper airway obstruction increased. Morphine treatment significantly decreased maximal inspiratory flow (Vᵢₘₐₓ) (from 3.4±0.4 ml/min to 2.3±0.5 ml/min, F_{2,2}=7.805; partial eta²=0.66; p<0.05) and minute ventilation during obstructed breathing (FIG. 11F, FIG. 11G and FIGS. 13A-13E). The effect of morphine was present during the entire 6 hour study (FIGS. 9A-9E and FIGS. 13A-13E).

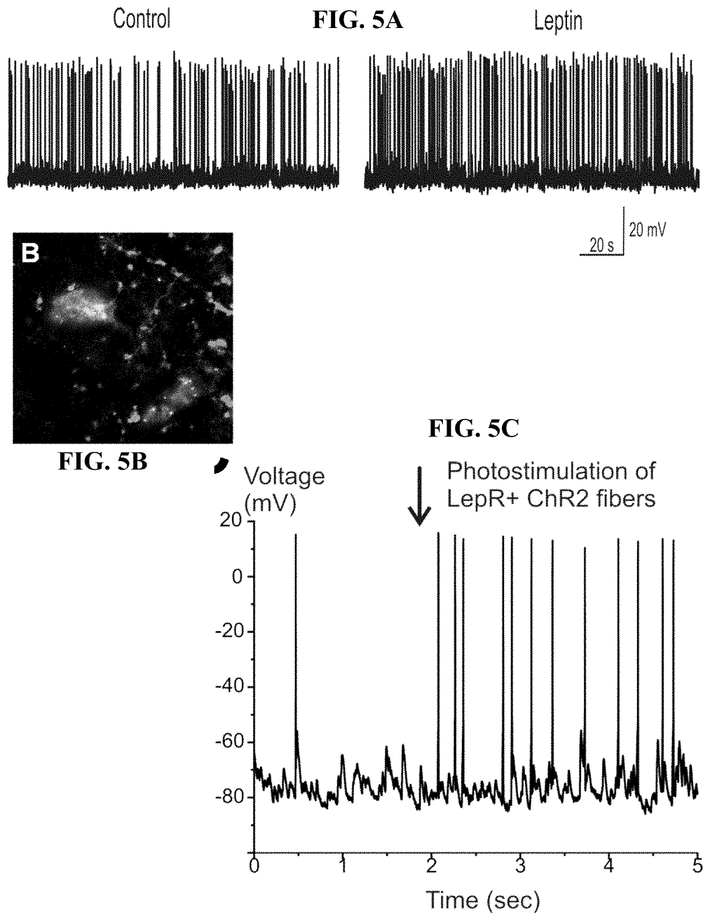

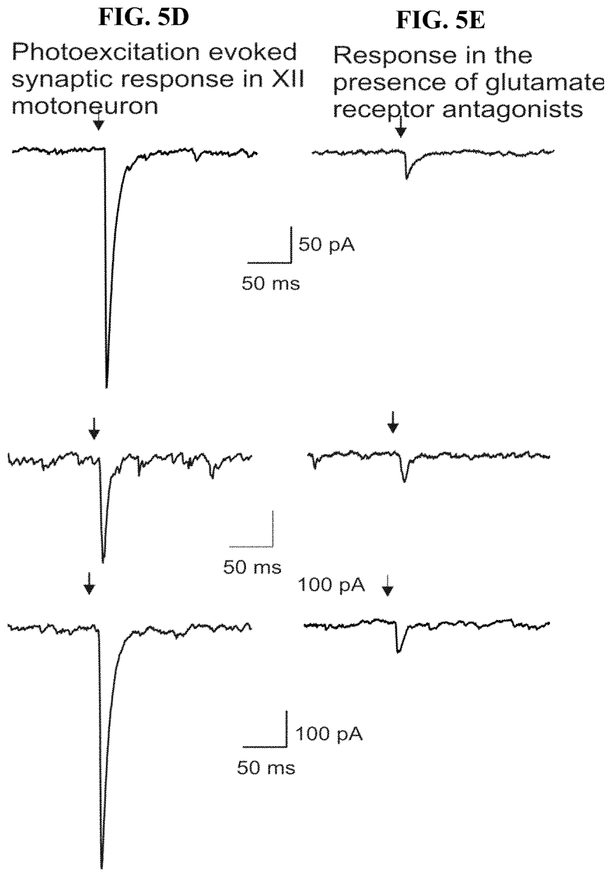

The above experiment was repeated in the same manner; however, in this experiment, male DIO mice were injected with IP morphine at 80 mg/kg instead of 10 mg/kg. FIG. 4A shows that the sub-lethal morphine dose induced severe inspiratory flow limitation (IFL), which was markedly improved by IN leptin administration (FIG. 4B). Further, the high morphine dosage induced respiratory depression; however, administration of IN leptin prevented the morphine-induced respiratory depression (FIG. 4C).

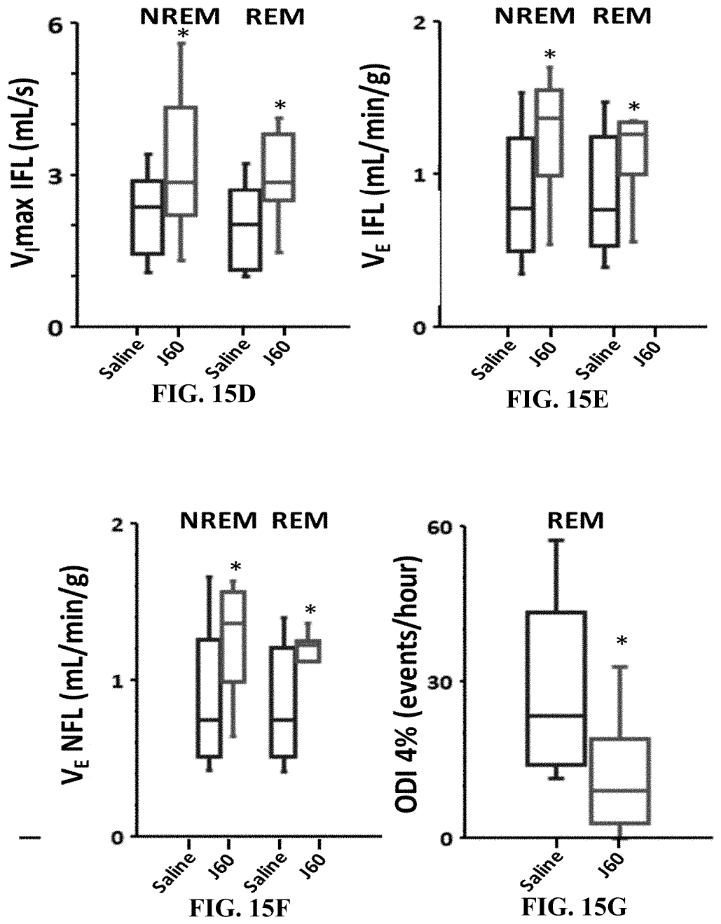

As ORS can lead to death, the effect of leptin in morphine lethality were tested on 51 lean male C57BL/6J mice. Mice received either intranasal leptin or intranasal vehicle (BSA) in a randomized manner. Thirty minutes after intranasal (IN) administration of leptin or vehicle, mice received 400 mg/kg of morphine IP and were be housed in cages according to the treatment received (leptin or vehicle). Mice were video monitored for 24 hours and survival time after morphine injection was recorded. The mice that survived were euthanized after the 24 hour observation period. As shown in FIG. 14, 23 of 25 mice treated with vehicle died compared to 18 out 26 mice treated with IN leptin (p=0.044). These data indicated that IN leptin improved mortality induced by morphine.

The data of this example show that morphine caused severe ORS and UAO and that IN leptin prevented morphine-induced ORS, UAO, hypoventilation, and mortality.

### EXAMPLE 4

### Mechanisms by which Leptin Prevents Opiate-Induced Decreases in HGN Activity

Studies were performed to establish the mechanisms by which focal application of leptin onto HGNs, as well as activation of LepR^{b}+ fibers that project to HGNs, prevented opiate-induced inhibition of HGNs, specifically fentanyl- and carfentanil-induced inhibition of HGNs.

First, a LepR^{b}−ChR2 mouse was generated by crossbreeding a LepR^{b}−Cre mouse with B6; 129S-Gt(ROSA)26Sor^{tm32(CAG-COP4*H134R/EYFP)Hze}/J mouse (JAX Stock #012569, ChR2 floxed). The resulting LepR^{b}+Cre mice allow for selective ChR2 optogenetic stimulation of LepR^{b}+ neurons for characterization of their synaptic neurotransmission to downstream targets, including hypoglossal motoneurons. Channelrhodopsin-2 (ChR2), an algal protein from Chlamydomonas reinhardtii, is a light-activated cation channel capable of inducing depolarization and action potentials in neurons. Expression of μ opiate receptor (MOR) and LepR^{b} on hypoglossal motoneurons (HGN) innervating genioglossus (GG) muscle was examined after injection of retrograde tracer cholera toxin B (CTB) to the GG muscle.

Results show that while HGNs do not express LepR^{b}, they are surrounded by LepR^{b}+ChR2+ fibers (FIG. 5B). Photoexcitation of ChR2 expressing fibers from LepR^{b}+ neurons evoked increases in firing in GG HGNs (FIG. 5C). Robust excitatory post-synaptic currents in HGNs evoked by photoexcitation of ChR2 expressing fibers from LepR^{b}+ neurons (FIG. 5D) were blunted, but not blocked by application of NMDA and non-NMDA glutamate receptor antagonists 50 μM of APV ((2R)-amino-5-phosphonovaleric acid; (2R)-amino-5-phosphonopentanoate; also referred to as AP5) and 50 μM of CNQX (6-cyano-7-nitroquinoxaline-2,3-dione; also referred to as cyanquixaline) (FIG. 5E). These data show that LepR^{b}+ neurons were (1) excited by leptin, (2) glutamatergic and (3) there were monosynaptic excitatory connections from LepR^{b}+ neurons to HGNs.

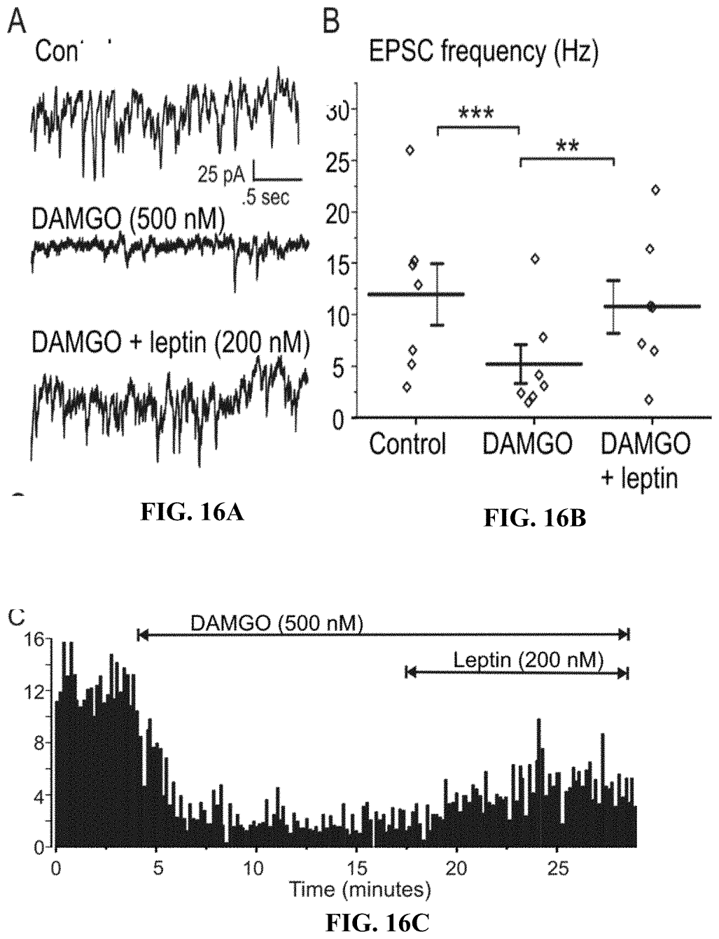

MOR (μ opiate receptor) agonists, such as fentanyl and carfentanil, suppressed the activity of HGNs in a manner not mediated via a change in muscarinic receptor activation or post-synaptic changes in HGN membrane properties. Rather, the opioid inhibition was mediated predominantly by presynaptic inhibition of excitatory glutamatergic neurotransmission to HGNs. HGNs received a major burst of excitatory inputs during inspiration that was critical for keeping the tongue forward and airway open during inspiration. Examples herein showed that morphine induces upper airway obstruction (UAO) reversed by leptin in vivo. To examine if leptin acts directly on the HMNs, retrograde tracer pseudorabies virus (PRV) was injected in the genioglossus muscle of LepR^{b}-GFP mice. Histology showed no evidence of LepR^{b} in the XII nucleus (FIG. 15A). Although HMNs do not appear to express LepR^{b}, HMNs were shown enmeshed in LepR^{b} (+) fibers (FIG. 15B). Next, excitatory Cre-dependent designer receptor exclusively activated by designer drugs (DREADDs) rAAV5.2-hSyn-DIO-hM3(Gq)-mCherry was placed in the nucleus of the solitary tract (NTS) of LepR^{b}−Cre mice (FIG. 15C). 4 weeks later, sleep studies were performed as described in the examples herein in the same mice treated either with IP saline or J60 (the DREADD ligand). J60 relieved UAO (FIGS. 15D and 15E) and up-regulated control of breathing (FIG. 15F), which resulted in resolution of sleep apnea (FIG. 15G). Thus, leptin stimulates breathing and upper airway patency acting in the NTS.

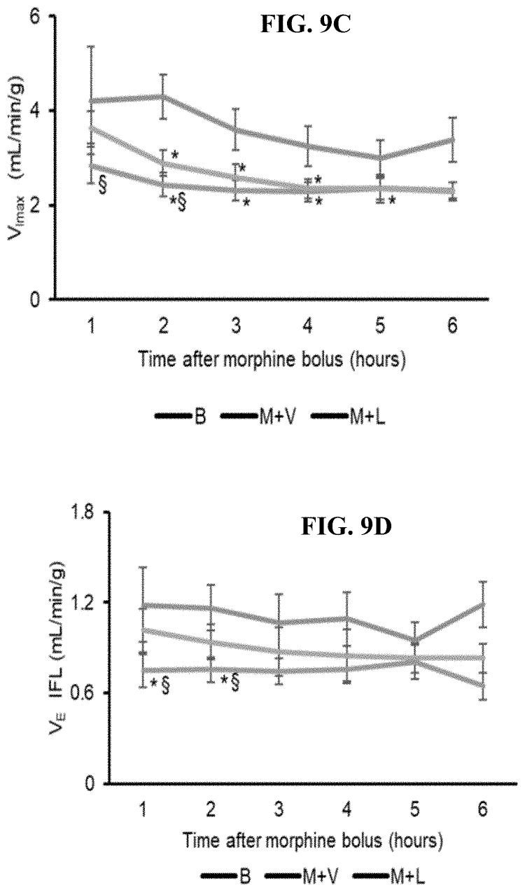

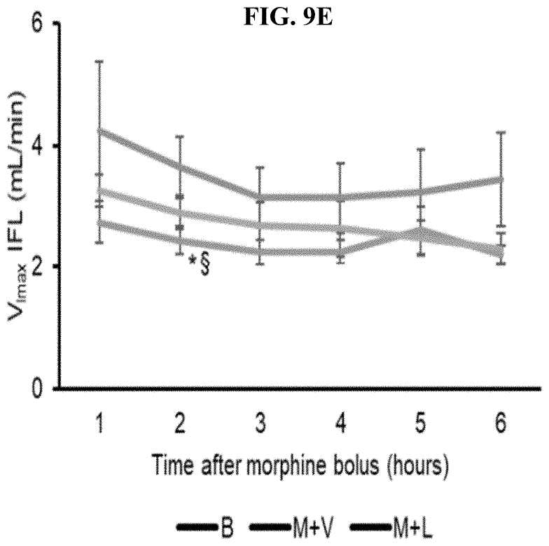

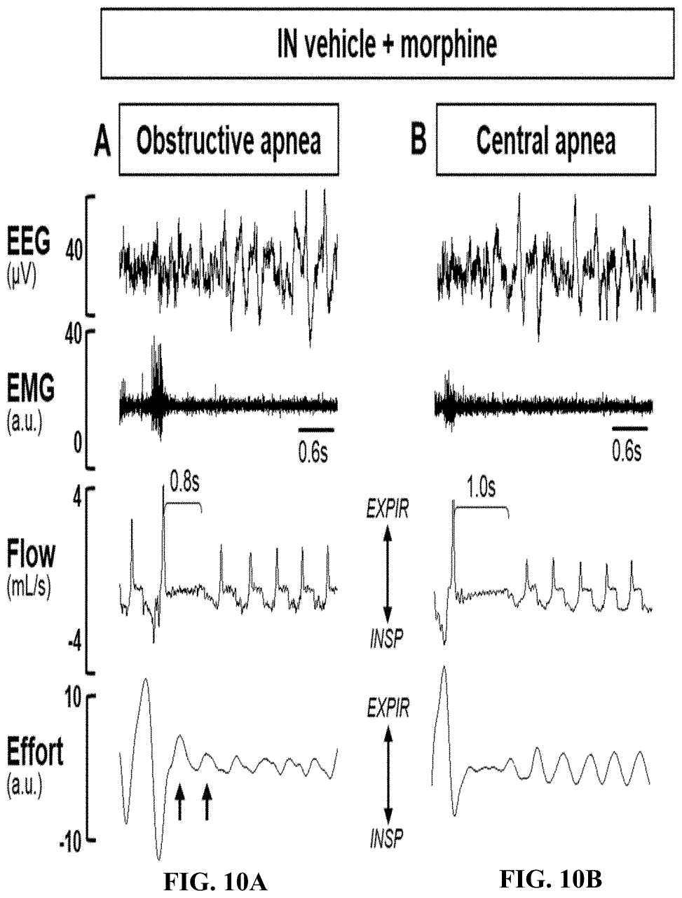

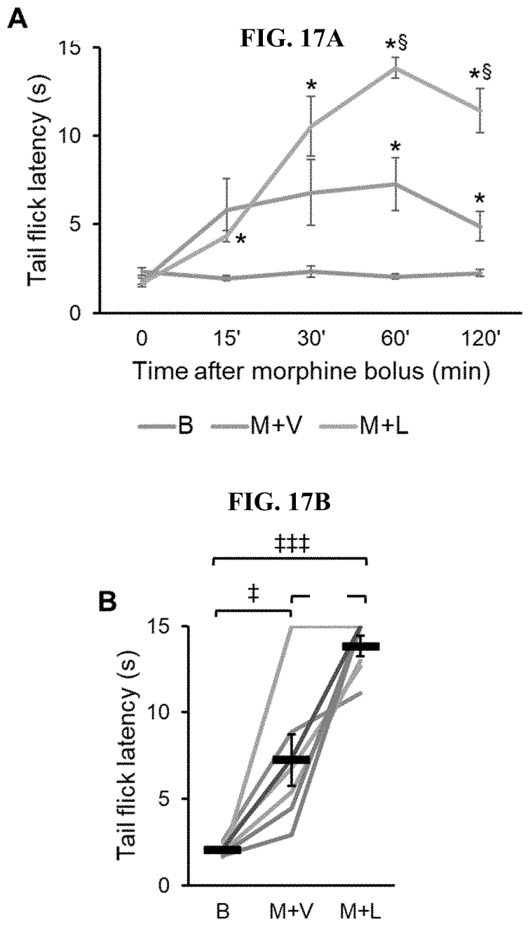

FIGS. 6A-6C show traces of HGN firing with each fictive inspiratory burst recorded from the XII nerve in a “bursting slice” in-vitro preparation. FIGS. 6D-6F show the average tracings recorded from 3 preparations. These preparations retained putative fictive inspiratory rhythms and HGNs increased firing with each burst. DAMGO ([D-Ala², N-MePhe⁴, Gly-ol]-enkephalin) is a synthetic opioid peptide with high μ-opioid receptor specificity. Prior to DAMGO, each burst evoked a barrage of typically 3-5 action potentials in HGNs (FIGS. 6A-6). DAMGO (500 nM) inhibited this activation, with typically only 2-3 action potentials associated with each burst. Leptin (200 nM) partially restored HGN activity, with bursting typically evoking 3-4 action potentials during each burst (FIG. 6G). DAMGO rapidly depressed spontaneously excitatory post-synaptic currents (EPSCs) in HGNs, and this adverse response was successfully reversed and treated with leptin (FIG. 7). Taken together, data showed that opioid mediated reductions in the activity of HGNs was caused by reduced excitatory drive to these neurons. These unexpected data show that leptin can reverse the inhibition of HGN activity. One source of excitatory drive to HGNs, based on the data herein (FIGS. 5A-5E), was from LepR^{b}+ neurons, as photoactivation of ChR2-expressing LepR^{b}+ fibers elicited an excitatory, predominantly glutamatergic, synaptic neurotransmission to HGNs. In addition, focal application of leptin to HGNs partially reversed opioid induced inhibition of HGN firing (FIG. 6) and EPSC suppression (FIG. 7). FIGS. 16A-16C show results from a similar experiment, this time using a focal application of 200 nM leptin to restore EPSC frequency

### EXAMPLE 5

### Intranasal Leptin Treatment of Fentanyl-Induced ORS

To assess the effectiveness of IN leptin treatment of fentanyl-induced ORS, sleep studies are performed as described in the examples above. Briefly, lean male (25-27 g) and female (23-25 g) and DIO male (43-47g) and DIO female (38-42 g) C57BL/6J mice are head-mounted, acclimated and baseline respiratory measurements collected. After collecting baseline measurement, all animals are treated with IP fentanyl at a dose that induces 50% ORS (decreasing minute ventilation by about 50) at 10 AM. Immediately after fentanyl treatment, IN leptin is administered at 0 (vehicle), 0.2, 0.4, 1, 2 or 5 mg/kg, or IN vehicle (BSA) as described in the examples above. The study is repeated one week later with leptin or vehicle in a cross-over fashion.

Pharyngeal collapsibility plays a pivotal role in the pathogenesis OSA and UAO under sedation, as reflected by increased upper airway collapsibility (Pcrit). Leptin decreased upper airway collapsibility in mice. A subset of mice instrumented under 1-2% isoflurane is monitored for upper airway pressure-flow relationships with head-out plethysmography while nasal pressure is ramped down from about 5 cm to about 20 cm H₂O over several breaths as described in Nishimura et al., Front Neurol 9, (2018), the disclosure of which is incorporated herein in its entirety. Pcrit measurements are performed at baseline. Fentanyl is administered at a dose that induces 50% ORS (decreasing minute ventilation by about 50) followed by IN leptin at either 0, or 0.2, 0.4, 1, 2 or 5 mg/kg and the measurements repeated.

LepR^{b} signaling, as measured by the amount of STAT3 phosphorylation, is determined in the lean and DIO mice used for the sleep studies and control of breathing measurements described about. The animals are treated with IN leptin or IN vehicle (BSA) as described in the previous examples herein. Fentanyl administered at a dose that induces 50% ORS or saline is injected IP. Mice are then sacrificed one hour later under 2% isoflurane. Brains are collected and subjected to cryosectioning and immunostaining for detection of pSTAT3 as an assessment of LepR^{b} signaling following methods described in the previous examples here.

### EXAMPLE 6

### Intranasal Leptin Treatment of Carfentanil-Induced ORS

To assess the effectiveness of IN leptin treatment of carfentanil-induced ORS, sleep studies are performed as described in the examples above. Briefly, lean male (25-27 g) and female (23-25 g) and DIO male (43-47g) and DIO female (38-42 g) C57BL/6J mice are head-mounted, acclimated and baseline respiratory measurements collected. After collecting baseline measurement, all animals are treated with IP carfentanil at a dose that induces 50% ORS (decreasing minute ventilation by about 50) at 10 AM. Immediately after carfentanil treatment, IN leptin is administered at 0 (vehicle), 0.2, 0.4, 1, 2 or 5 mg/kg, or IN vehicle (BSA) as described in the examples above. The study is repeated one week later with leptin or vehicle in a cross-over fashion.

A subset of mice instrumented under 1-2% isoflurane is monitored for upper airway pressure-flow relationships with head-out plethysmography while nasal pressure is ramped down from about 5 cm to about 20 cm H₂O over several breaths as described in Nishimura et al., Front Neurol 9, (2018), the disclosure of which is incorporated herein in its entirety. Pcrit measurements are performed at baseline. Carfentanil is administered at a dose that induces 50% ORS (decreasing minute ventilation by about 50) followed by IN leptin at either 0, or 0.2, 0.4, 1, 2 or 5 mg/kg and the measurements repeated.

LepR^{b} signaling, as measured by the amount of STAT3 phosphorylation, is determined in the lean and DIO mice used for the sleep studies and control of breathing measurements described about. The animals are treated with IN leptin or IN vehicle (BSA) as described in the previous examples herein. Carfentanil administered at a dose that induces 50% ORS or saline is injected IP. Mice are then sacrificed one hour later under 2% isoflurane. Brains are collected and subjected to cryosectioning and immunostaining for detection of pSTAT3 as an assessment of LepR^{b} signaling following methods described in the previous examples here.

### EXAMPLE 7

### Intranasal Leptin and Naloxone Treatment of Opioid-Induced ORS

Naloxone has a short half-life and may be insufficiently effective for treatment of opioid overdose, particularly in events where opioids have been weaponized. Leptin half-life is significantly longer than naloxone. To examine the effectiveness of administering IN leptin with naloxone in treating morphine-, fentanyl-, and carfentanil-induced ORS the system of respiratory recording, described in the previous examples, analyzes control of breathing and upper airway function in both lean and obese mouse models. Experiments described in examples 1-6 are frepeated in the same manner with the exception that following either morphine, fentanyl, or carfentanil administration at sub-let chal doses, lean and obese mice are treated with placebo, naloxone (1 mg/kg) only or naloxone (1 mg/kg)+IN leptin from 0.2 to 5 mg/kg. Mixed-effect multivariable linear regression models are developed that examine the different respiratory parameters as a function of leptin, naloxone, or leptin+naloxone treatment of opioid-induced ORS compared to untreated opioid-induced ORS. Because leptin and naloxone target different receptors after crossing the blood brain barrier, the IN leptin+naloxone combination is more effective compared to leptin or naloxone alone. The combination treatment of IN leptin+naloxone may have additive or synergistic effects depending on the particular treatment parameters and targeted subject.

### EXAMPLE 8

### Synaptic Mechanisms by which Leptin Prevents Opioid-Induced Induced Decreases in HGN Activity

Focal application of leptin to HGNs reverses fentanyl and carfentanil mediated inhibition of excitatory neurotransmission to HGNs and HGN activity. While not selective for the fibers of origin, these experiments (using the ex-vivo system described in example 4 above) test for leptin receptor mediated restoration of excitatory synaptic inputs to HGNs in neonatal animals in which slices maintain putative fictive respiratory bursts, and well as in older animals (devoid of putative respiratory bursts). A range of concentrations of fentanyl and carfentanil applied to the fibers establish the dose response relationship and establish the concentration that elicits a 50% inhibition of synaptic transmission and firing of HGNs. Initial doses tested include: (1) fentanyl at 0.01, 0.1, 1, 10, 100 microM; and (2) carfentanil at 0.1, 1, 10, 100 nM and 1 microM. These doses can be revised and refined as necessary to obtain a well-defined dose-response relationship and 50% effective dose. Once a concentration of fentanyl and carfentanil that elicits a 50% inhibition of synaptic transmission and firing of HGNs is determined, increasing concentrations of leptin are applied to treat fentanyl- or carfentanil-mediated inhibition of synaptic transmission and firing of HGNs. The range of leptin concentrations used are 0.1, 1, 10, 100 and 500 nM, although this concentration can be optimized as needed. Each of these solutions is ejected from a puffer pipette positioned within 30 μm from the patched HGN localizing the site of action to HGNs and their surrounding synaptic contacts—preventing any off-target or poly-synaptic effects (such as changes in bursting frequency due to inhibition of neurons in the preBotzinger complex). The sequence of administration follows a realistic therapeutic treatment paradigm where the dose of fentanyl and carfentanil that elicits a 50% reduction of HGN activity is administered first followed by leptin administration. Additional experiments are performed in the presence of TTX (1 microM) to block synaptic neurotransmission and isolate miniature EPSCs (mEPSCs). Additionally, a concentration of leptin that is 50% effective in treating fentanyl- and carfentanil-induced inhibition of synaptic transmission and firing of HGNs is added to the fibers in addition to naloxone to access the effects of the combined treatment on the underlying mechanisms of action.

The activation of LepR^{b}+ fibers as a suppressor of fentanyl- and Z carfentanil-induced decreases in excitatory neurotransmission to HGNs and HGN neuronal firing is assessed using the same ex-vivo model described above. Using this model, the origin of the LepR^{b}+ fibers involved is also determiend. Injecting a floxed ChR2 virus (AAV1-EF1a-DIO-hChR2) into either the DMH or NTS in LepR^{b}−Cre mice selectively expresses ChR2 in LepR^{b}+ neurons originating from the targeted nuclei. After 3 weeks of recovery, ChR2 fibers from these neurons are identified in the brainstem and photoactivated for studying neurotransmission from LepR^{b}+ neurons to HGNs identified by retrograde tracers injected into the GG. Photoactivation of these fibers is measured to assess attenuation of fentanyl- and carfentanil-induced inhibition of synaptic neurotransmission and HGN firing activity. These experiments are performed using a range of concentrations of fentanyl and carfentanil to establish the dose response relationship and establish the concentration that elicits a 50% inhibition of synaptic transmission and firing of HGNs. For example, fentanyl concentrations range from about 0.01, 0.1, 1, 10, 100 microM, and doses of carfentanil concentrations range from about 0.1, 1, 10, 100 nM and 1 microM. Next, photoactivation of DMH LepR^{b}+synapses is assessed for restoration of EPSC frequency, amplitude, and HGN firing. ChR2 fibers are photoexcited with a Crystalaser (473 nm, 10 mW). Single, paired (0.1 to 10 Hz), and bursting patterns of photostimulation are performed (from 0.1 Hz to 10 Hz, for durations of 100 ms to 1 second) to excite DMH LepR^{b}+ synaptic terminals that surround HGNs and evoked synaptic currents and changes in firing are recorded using both the whole cell voltage clamp and current clamp configurations. Similar experiments are performed by photoactivating NTS LepR^{b} synapses.

Photoexcitation of LepR^{b}-ChR2 expressing fibers from DMH LepR^{b}+ neurons and NTS LepR^{b}+ neurons can attenuate the fentanyl- and carfentanil-induced inhibition of EPSCs and firing in HGNs.

### EXAMPLE 9

### Leptin and Opioid-Induced Anesthesia

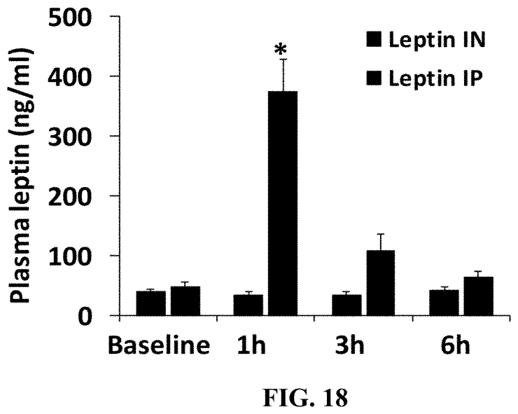

In order to examine if leptin improves breathing without compromising analgesia, we performed the tail flick test, a validated test of pain perception in a cross-over randomized manner. Briefly, for measurement of tail flick latencies after tail immersion in a hot water bath, DIO mice were acclimated to restrainer tube for 3 days prior to test. All measures were recorded between 13:00-17:00. DIO Mice were immobilized (25-30 seconds) in an acrylic tube and the distal ⅓ of the tail was immersed in water at 50±1° C. Nociceptive latency was recorded the moment a tail flick was observed. A maximum of 15 seconds immersion time was used to avoid tissue damage. Baseline latencies were determined prior to any intervention and measured twice with 5 minutes interval between measures. Baseline values shown represent the mean of the two measurements acquired. After baseline measures, DIO mice received IN vehicle or IN leptin (0.8 mg/kg) in a crossover randomized manner followed by IP morphine (10 mg/kg), 30 minutes after IN treatment. Tail flick latencies were measured 15, 30, 60, and 120 minutes after IP morphine/saline administration. FIGS. 17A and 17B show the increase in tail flick latency after morphine administration. IN leptin increased tail flick latency measured 60 min and 120 min after morphine bolus (FIG. 17A) and was consistent in six out of seven tested mice (FIG. 17B). These data showed that leptin did not decrease analgesia in morphine treated mice.

### EXAMPLE 10

### Subcutaneous Leptin Increases Blood Pressure (BP) in Mice Acting in the Carotid Bodies

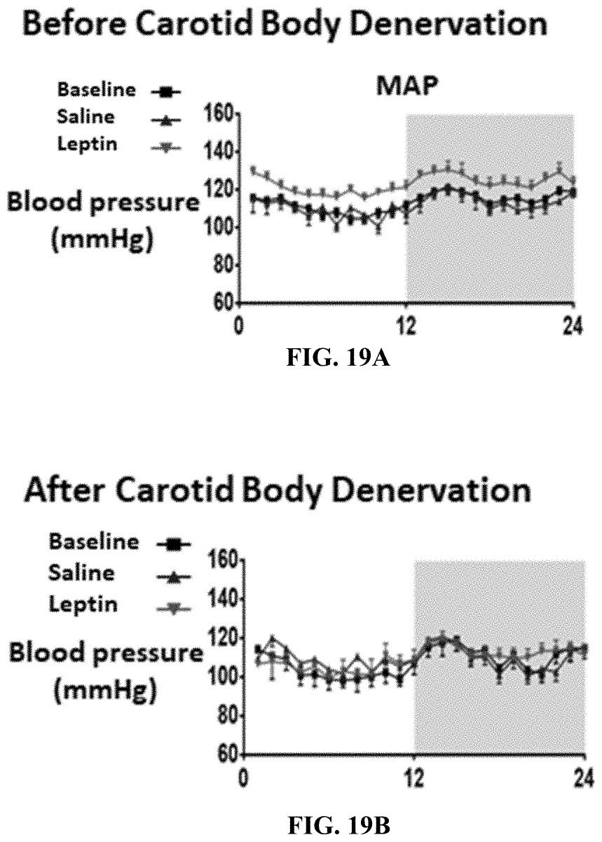

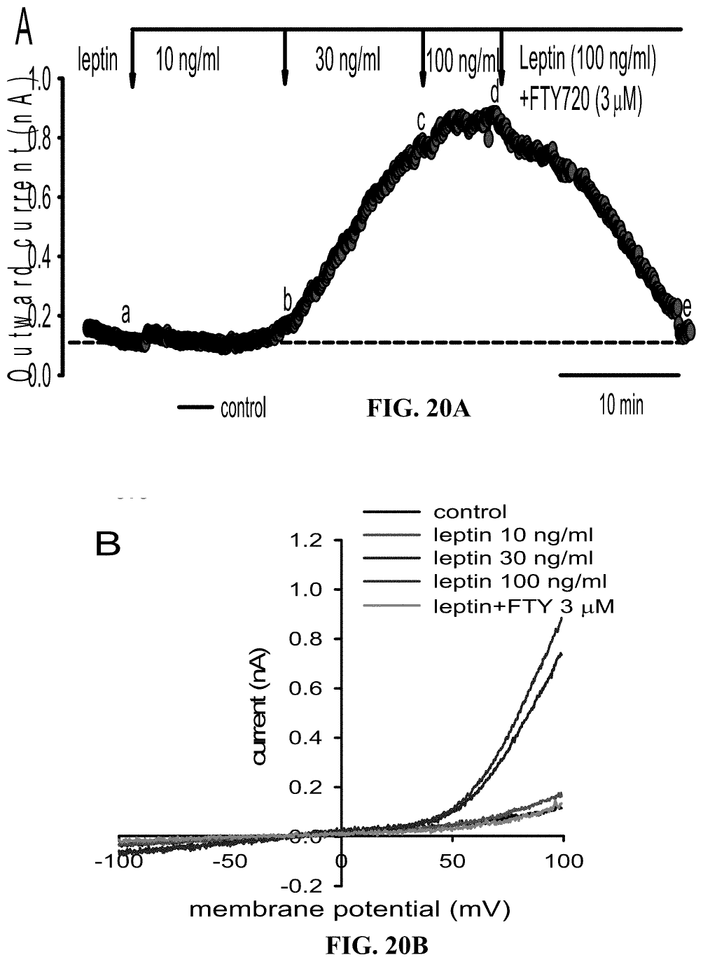

Blood pressure was measured by telemetry (DSI) implanted in the left femoral artery of freely behaving lean C57BL/6J mice at baseline and during leptin infusion (120 μg/day for 3 days via a SC pump) before and after carotid body (CB) denervation or sham surgery. As shown in FIG. 19A SC Leptin increased blood pressure by 13 mm Hg during the day (9 am-9 pm, light phase) and by 16 mm Hg at night and this effect was completely abolished by CB denervation, but not by sham surgery as shown in FIG. 19B. These data suggest that leptin acted peripherally (in CB) to increase BP. Because IN leptin did not increase plasma leptin levels (FIG. 18), it does not increase BP.

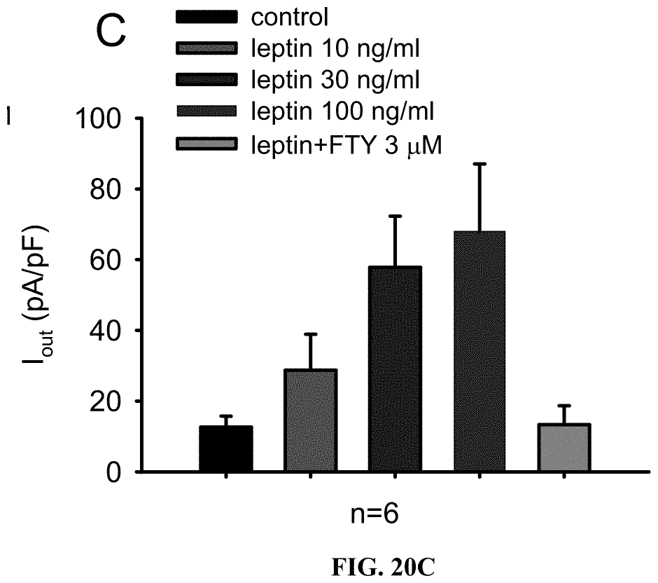

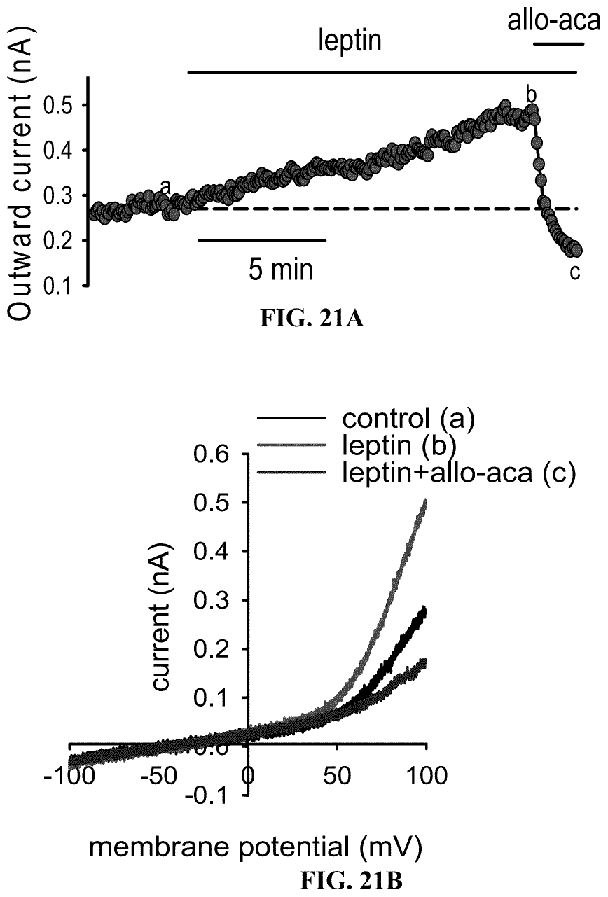

Leptin activates Trpm7 in the glomus cells of the carotid body. Because of this, a bioassay was developed using LepR^{b} expressing pheochromocytoma PC12 (PC12^{LEPRb}) cells. Non-selective cation current was recorded using amphotericin-B perforated-patch in the absence of extracellular and intracellular K⁺ (replaced by Cs) and Mg²⁺, after VDCC and Cl⁻ currents were inhibited by nifedipine, 4,4′-diisothiocyanatostilbene-2,2′-disulphonic acid (DIDS), and niflumic acid. Voltage-ramps elicited an outward rectifying current that resembled Trpm7 current reported in other cells. Application of leptin at concentration of 10-100 ng/ml activated a concentration-dependent increase in the non-selective cation channel current, which was completely blocked by the Trpm7 antagonist FTY720 (FIGS. 20A-20C). The effect of leptin was also completely blocked by 300 nM of the specific leptin-receptor antagonist Allo-aca (FIGS. 21A and 21B), indicating that the current was activated via LepR^{b}. Using this assay, functional activity of anti-leptin antibody, for example, can be assessed.

1. A method of treating opioid-induced respiratory depression in a subject in need thereof, the method comprising administering leptin.

2-6. (canceled)

7. The method of claim 1, wherein the human subject in need thereof has leptin resistance, has obstructive sleep apnea, or has a combination thereof.

8. (canceled)

9. The method of claim 1, wherein the breathing rate of the subject in need thereof increases after administering leptin compared to an untreated subject with identical disease condition and predicted outcome.

10. (canceled)

11. The method of claim 1, wherein the upper airway patency of the subject in need thereof increases after administering leptin compared to an untreated subject with identical disease condition and predicted outcome.

12. (canceled)

13. The method of claim 1, wherein the obstructive sleep apnea of the subject in need thereof improves after administering leptin compared to an untreated subject with identical disease condition and predicted outcome.

14. (canceled)

15. The method of claim 1, wherein the tidal volume of the subject in need thereof increases after administering leptin compared to an untreated subject with identical disease condition and predicted outcome.

16. (canceled)

17. The method of claim 1, wherein the maximum inspiratory flow rate (Vᵢₘₐₓ) of the subject in need thereof increases after administering leptin compared to an untreated subject with identical disease condition and predicted outcome.

18. (canceled)

19. The method of claim 1, wherein the minute ventilation of the subject in need thereof increases after administering leptin compared to an untreated subject with identical disease condition and predicted outcome.

20-24. (canceled)

25. The method of claim 1, wherein leptin is administered in combination with at least one opioid.

26. The method of claim 25, wherein leptin is administered in combination with at least one opioid for up to 5 days following surgery.

27. The method of claim 25, wherein leptin is administered immediately following an overdose of at least one opioid.

28. A composition for treating opioid-induced respiratory depression in a subject in need thereof, the composition comprising a therapeutically effective amount of leptin.

29. The composition of claim 28, wherein the composition further comprises at least one pharmaceutical excipient.

30. The compositions of claim 28, wherein the composition is formulated for intranasal administration.

31-33. (canceled)

34. The composition of claim 33, wherein the therapeutically effective amount of leptin comprises an amount that increases breathing rate of the subject in need thereof after administering the composition, increases upper airway patency of the subject in need thereof after administering the composition, or any combination thereof.

35. (canceled)

36. The composition of claim 28, wherein the leptin is a recombinant human leptin, a pegylated recombinant human leptin (PEG-OB), a recombinant human methionyl leptin, a leptin peptidomimetic, a biologically active fragment of leptin, a fusion peptide of leptin with an Fc fragment of immunoglobulin, a fusion peptide of the biologically-active fragment of leptin with the Fc fragment of immunoglobulin, a leptin agonist, or a combination thereof.

37. (canceled)

38. A method of treating opioid-induced respiratory depression in a subject in need thereof, the method comprising administering leptin intranasally,
wherein the subject in need thereof is a human subject having leptin resistance, having obstructive sleep apnea, having an upper airway obstruction, or any combination thereof.

39-45. (canceled)

46. The method of claim 38, wherein leptin is administered intranasally at least once, at least once every 6 hours as needed, or at least once 12 hours as needed to the subject in need thereof.

47-48. (canceled)

49. The method of claim 38, wherein leptin is acutely administered intranasally to the subject in need thereof.
